(19) United States
(12) Patent Application Publication
(10) Pub. No.: US 2025/0137929 A1
(43) Pub. Date: May 1, 2025

(54) DEVICE, SYSTEM, AND METHOD OF DETECTING BIOLOGICAL AGENTS

(71) Applicant: University of Georgia Research Foundation, Inc., Athens, GA (US)

(72) Inventors: Yiping Zhao, Bogart, GA (US); Ralph A. Tripp, Watkinsville, GA (US); Yanjun Yang, Athens, GA (US)

(21) Appl. No.: 18/496,455

(22) Filed: Oct. 27, 2023

(51) Int. Cl. G01N 21/552; G01N 21/25; G01N 33/543

(52) U.S. Cl. CPC G01N 21/554; G01N 21/25; G01N 33/54373; G01N 2021/258

(57) ABSTRACT

The present disclosure provides for devices and systems for detecting biological agents (e.g., virus such as coronavirus) using nanotriangles having a capture agent. The present disclosure also includes methods of detecting the presence and amount of the biological agent in a sample, such as saliva, blood, or urine. In addition, the present disclosure provides for a detecting module and a method of making the detecting module, where the detecting module includes the nanotriangles having the capture agent bonded thereto that has an affinity for the biological agent.

### BACKGROUND

The SARS-COV-2 pandemic COVID-19 disease has led to unprecedented burden on national and international healthcare. This has motivated researchers to develop reliable tools to aid SARS-CoV-2 diagnostics. Based on current limitations, there is a need for new diagnostic techniques and systems.

### SUMMARY

The present disclosure provides for devices and systems for detecting biological agents (e.g., virus such as coronavirus) using nanotriangles having a capture agent. The present disclosure also includes methods of detecting the presence and amount of the biological agent in a sample. In addition, the present disclosure provides for a detecting module and a method of making the detecting module, where the detecting module includes the nanotriangles having the capture agent bonded thereto that has an affinity for the biological agent.

The present disclosure provides for a device for detecting a biological agent, comprising: a white light source; a UV-Vis-NIR spectrometer; and a detecting module comprising a substrate including a plurality of nanotriangles having a capture agent disposed on the nanotriangle, wherein the capture agent has an affinity for a biological agent, wherein the biological agent is a type of virus or a type of bacteria, wherein the white light source, the UV-Vis-NIR spectrometer, and the detecting module are interfaced so that the light from the white light source is configured to be directed towards the substrate of the detecting module, wherein the UV-Vis-NIR spectrometer is configured relative to the detecting module to receive a signal from the nanotriangles as a result of the light from the white light source interacting with the nanotriangles.

The present disclosure provides for a method of detecting a biological agent, wherein the method includes: disposing a sample of a fluid onto an area of the detecting module as described above and herein, wherein prior to disposing the sample, the detecting module includes a capture agent bonded to the nanotriangle and wherein a first localized surface plasmon resonance (LSPR) signal is known that corresponds to the nanotriangle bonded to the capture agent; and detecting a second LSPR signal using the UV-Vis-NIR spectrometer after disposing the sample in the first area, wherein when the second LSPR signal is different than the first LSPR signal, this indicates that the biological agent is bonded to the capture agent, which indicates that the biological agent is present in the sample.

The present disclosure provides for a system for detecting a biological agent, comprising: a white light source; a UV-Vis-NIR spectrometer; and a detecting module, wherein the white light source is configured to direct a white light at the detecting module, wherein the UV-Vis-NIR spectrometer is configured to detect a signal corresponding to nanotriangle present in the detecting module; wherein the detecting module includes a substrate including a plurality of the nanotriangles having a capture agent disposed on the nanotriangle, wherein the capture agent has an affinity for a biological agent, wherein the biological agent is a type of virus or a type of bacteria, wherein prior to the introduction of sample of fluid to the detecting module, the UV-Vis-NIR spectrometer is configured to detect a first localized surface plasmon resonance (LSPR) signal that corresponds to the nanotriangle having the capture agent, wherein after introduction of the sample of fluid that contains the biological agent, the UV-Vis-NIR spectrometer is configured to detect a second LSPR signal that corresponds to the nanotriangle having the capture agent and the biological agent bonded to the capture agent, wherein the first LSPR signal and the second LSPR signal are different, wherein the difference corresponds to the amount of biological agent present in the sample.

The present disclosure provides for a method of making a detecting module, comprising: providing a substrate having an array of addressable nanotriangles, introducing a capture agent to the substrate including the array of addressable nanotriangles, wherein the capture agent bonds to the nanotriangles, removing the captures agents not bonded to the addressable nanotriangles, introducing a second agent to the substrate including the addressable nanotriangles, wherein the addressable nanotriangles that do not have a capture agent bonded to it will bond with the second agent; and removing the second agents not bound to the addressable nanotriangles.

### BRIEF DESCRIPTION OF THE DRAWINGS

The presently disclosed subject matter will be better understood, and features, aspects and advantages other than those set forth above will become apparent when consideration is given to the following detailed description thereof. Such detailed description makes reference to the following drawings.

FIG. 1A illustrates a proposed strategy for spike RBD protein or virus detection. Step 1: AgNT array fabrication. Step 2: ACE2 immobilization. Step 3: BSA blocking. Step 4: spike RBD protein or CoV detection. FIG. 1B illustrates the changes of transmission spectrum from AgNT array sensor before and after detection.

FIG. 2A illustrates the plot of the shift of LSPR wavelength Δλ of AgNT array versus the different concentration C_{ACE2} of ACE2 protein. FIG. 2B illustrates the transmission spectra of AgNT array at different stages of surface functionalization and detection: immobilized with ACE2 protein, BSA blocking, spike RBD protein detection at C_{spikeRBD}=675 pM as well as AgNT array sensor incubating with dilution buffer. FIG. 2C illustrates the corresponding changes in LSPR wavelength Δλ and transmission intensity 1750 at each step.

FIG. 3A illustrates the concentration-dependent transmission spectra for C_{spikeRBD}=0.38 pM to 9420 pM. FIG. 3B illustrates the semi-log plots of the LSPR transmission intensity I₇₅₀ versus C_{spikeRBD} and the LSPR wavelength shift Δλ_{spikeRBD} versus C_{spikeRBD}. The solid lines are the fitting result.

FIG. 4A illustrates the transmission spectra of the AgNT wells treated with DMEM buffer (with 1% FBS), 229E, OC43, and NL63 at the concentration of 10⁵ PFU/mL, and FIG. 4B illustrates the corresponding I₇₅₀ and Δλ plot. FIG. 4C illustrates the concentration-dependent transmission spectra of NL63 detection ranging from 625 to 10⁴ PFU/mL in untreated saliva. FIG. 4D illustrates the corresponding semi-log plot of I₇₅₀ versus C_{NL63} and semi-log plot of Δλ_{NL63} versus C_{NL63}.

FIG. 5A illustrates the scheme of coronavirus detection model. FIG. 5B illustrates the semi-log plot and linear fitting of the virus particles on one AgNT versus C_{NL63}.

FIG. 6 illustrates the time-dependent detection of spike RBD (circles) at C_{spikeRBD}=2020 pM and NL63 (triangles) at C_{NL63}=12500 PFU/mL. The solid curves are fitting results based on Eq. 8.

FIG. 7A illustrates a diagram of the AgNT array fabrication procedure. FIG. 7B illustrates a photo of PDMS wells on AgNT array, i.e., AgNT wells.

FIG. 8A illustrates a representative AFM image and FIG. 8B illustrates a transmission spectrum of AgNT array. Refractive index sensing performance characterization of the AgNT array: FIG. 8C illustrates the RI-dependent UV-vis transmission spectra, and FIG. 8D illustrates the plot of do versus RI.

FIG. 9 illustrates the transmission spectra of 8 AgNT wells. The average λ₀=641±2 nm.

FIG. 10 illustrates the transmission spectra of AgNT incubating in PBS and phosphate buffer.

FIG. 11 illustrates the LSPR wavelength shift Δλ_{ACE2} of 20 ACE2 immobilized AgNT wells.

FIGS. 12A and 12B illustrate the optimized buffer for spike RBD protein detection. Here, “PB” refers to phosphate buffer and “dilution” is dilution buffer.

FIG. 13 illustrates a typical SERS spectrum of ACE2 immobilization on the AgNR substrate and peak identification.

FIGS. 14A-14C illustrate the stability test of the AgNT array sensor stored in sealed opaque pouches and kept at 20° C. FIG. 14A illustrates the transmission spectra of AgNT array sensors after stored for zero (fresh), one, and three weeks. FIG. 14B illustrates the transmission spectra of AgNT array sensors treated with 2020 pM spike RBD protein after stored for zero (fresh), one, and three weeks. FIG. 14C illustrates the plots of the corresponding redshifts Δλ_{spikeRBD} for different storage period.

FIGS. 15A and 15B illustrate the reproducibility test of five batches of AgNT array sensors for spike RBD protein detection. FIG. 15A illustrate the transmission spectra of 5 batches of AgNT array sensors treated by 2020 pM spike RBD protein. FIG. 15B illustrates the plots of the corresponding Δλ_{spikeRBD}.

FIGS. 16A and 16B illustrate the reproducibility test of 5 batches of AgNT array sensor for CoV NL63 detection. FIG. 16A illustrates the transmission spectra of 5 batches of AgNT array sensors treated by 50000 PFU/mL CoV NL63. FIG. 16B illustrates the plots of the corresponding LSPR shifts Δλ_{NL63}.

FIG. 17 illustrates the scheme of coronavirus detection model. Case I: the outside of the AgNT is only air. Case II: the outside of the AgNT is coating with a ACE2 protein layer. Case III: after CoV NL63 detection, the outside of the AgNT is coating with ACE2 and NL63 layers.

FIG. 18A illustrates the semi-log plot of the effective RI versus the concentrations of CoV NL63 C_{NL63}. FIG. 18B illustrate the semi-log plot of the volume fraction δᵢ versus C_{NL63}. The red lines represent the best linear fits.

FIG. 19 illustrates Table S1.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, materials science, mechanical engineering, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by volume, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequences where this is logically possible.

It must be noted that, as used in the specification and the appended claims, the singular forms “a,” “an,” and “the” include plural referents unless the context clearly dictates otherwise. Thus, for example, reference to “a compound” includes a plurality of compounds. In this specification and in the claims that follow, reference will be made to a number of terms that shall be defined to have the following meanings unless a contrary intention is apparent.

The term “array” encompasses the term “microarray” and refers to an ordered array presented for binding to biological agents and the like.

An “array” includes any two-dimensional or substantially two-dimensional (as well as a three-dimensional) arrangement of addressable regions including the nanotriangles and the like.

A substrate may carry one, two, four or more arrays disposed on a front surface of the substrate. Depending upon the use, any or all of the arrays may be the same or different from one another and each may contain multiple spots or features (e.g., a plurality of nanotriangles). A typical array may contain one or more, including more than two, more than ten, more than one hundred, more than one thousand, more ten thousand features, or even more than one hundred thousand features, in an area of less than about 20 cm² or even less than about 10 cm² (e.g., less than about 5 cm², including less than about 1 cm² or less than about 1 mm² (e.g., about 100 μm², or even smaller)). For example, features may have a longest dimension (e.g., between points of the triangle or other areas of the triangle that are furthest apart from one another) of about 50 to 2000 nm.

An array “package” may be the array plus a substrate on which the array is deposited, although the package may include other features (such as a housing with a chamber). A “chamber” references an enclosed volume (although a chamber may be accessible through one or more ports). It will also be appreciated that throughout the present application, that words such as “top,” “upper,” and ‘lower” are used in a relative sense only.

An array is “addressable” when it has multiple regions of the same or different features (e.g., nanotriangles) such that a region (i.e., a “feature” or “spot” of the array) at a particular predetermined location (i.e., an “address”) on the array can detect at that particular region.

A “scan region” refers to a contiguous (preferably, rectangular) area in which the array spots or features of interest, as defined above, are found or detected. The scan region is that portion of the total area queried from which resulting signal is detected and recorded.

An “array layout” refers to one or more characteristics of the features, such as feature positioning on the substrate, one or more feature dimensions, and an indication of a moiety at a given location.

### DISCUSSION

Embodiments of the present disclosure provide for devices and systems for detecting biological agents (e.g., virus such as coronavirus) using nanotriangles having a capture agent. The present disclosure also includes methods of detecting the presence and amount of the biological agent in a sample (e.g., a fluid such as saliva, blood, or urine). In addition, the present disclosure provides for a detecting module and a method of making the detecting module, where the detecting module includes the nanotriangles having the capture agent bonded thereto that has an affinity for the biological agent.

In particular and described in more detail in Example 1, the present disclosure provides for devices (e.g., an array), systems, and methods that can use a silver nanotriangle bonded to a human angiotensin-converting enzyme 2 protein (ACE-2) to detect corona virus (e.g., SARS-COV-2 spike RBD proteins and CoV NL63). A UV-Vis NIR spectrometer with a white light source (e.g., Ocean Optics USB-2000 and light source HL-2000) can be used to detect a shift of the localized surface plasmon (LSPR) signal (wavelength) corresponding to the presence of the coronavirus, where a high sensitivity and specificity of SARS-COV-2 spike RBD proteins and CoV NL63 are achieved. As discussed in detail in Example 1, the present disclosure provides for a rapid, portable, and cost-effective method to detect the infection of SARS-COV-2 is fundamental toward mitigating the COVID-19 pandemic. The present disclosure provides a human ACE2 functionalized silver nanotriangle array LSPR sensor for rapid coronavirus detection, which is validated by SARS-COV-2 spike RBD protein and CoV NL63 virus with high sensitivity and specificity. In an aspect and as described in Example 1, a linear shift of the LSPR wavelength and transmission intensity at a fixed wavelength (750 nm) versus the logarithm of the concentration of the spike RBD protein and CoV NL63 is observed. In an aspect, the limits of detection for the spike RBD protein, CoV NL63 in untreated saliva are determined to be 0.38 pM, and 625 PFU/mL, respectively, while the detection time is found to be less than 20 min. Thus, the AgNT array optical sensor could serve as a potential rapid point-of-care COVID-19 diagnostic platform for real sample analysis. Additional details are provided in Example 1 and the present disclosure is not limited to the aspects discussed in Example 1.

Now having provided a brief introduction, additional details are provided. The present disclosure provides devices (e.g., an array) that can be used for detecting biological agents such as a virus (e.g., coronavirus) or bacteria. In an aspect, the device includes a white light source and a UV-Vis-NIR spectrometer. The white light source can be any tungsten (or tungsten-halogen, or halogen) lamp or white light LED source. The portable UV-Vis-NIR spectrometer can be a device such as an Ocean Optics USB-2000, StellarNet Blue-Wave series, Tec5USA spectrometer modules, Hamamatsu mini-spectrometer series, or other similar spectrometer that is capable of measuring optical spectra in the wavelength range from 300 nm to 2,000 nm. In an aspect, the white light source, the UV-Vis-NIR spectrometer and the detecting module are interfaced so that the light from the white light source is configured to be directed towards the substrate of the detecting module. The UV-Vis-NIR spectrometer is configured relative to the detecting module to receive a signal from the nanotriangles as a result of the light from the white light source interacting with the nanotriangles. The device is configured to detect the presence of the biological agent within 20 minutes or less, or 15 minutes or less, or 10 minutes or less or 5 minutes or less (e.g., “or less” being about 30 seconds, about 1 minutes, about 2 minutes, or about 5 minutes) upon introduction of a sample to the detecting module, where the sample includes the biological agent. In an aspect, the UV-Vis-NIR spectrometer is configured to detect a change in the localized surface plasmon resonance (LSPR) of the nanotriangle with the capture agent bound to the nanotriangle and when the biological agent is bonded to the capture agent that is bonded to the nanotriangle. The difference can be quantified and corresponds to the amount of biological agent present in the sample, thereby allowing for precise and fast analysis of the sample, which would be highly beneficial during a pandemic.

In an aspect, the detecting module includes a substrate including a plurality of nanotriangles (e.g., 100s to 1000s to 100,000s or more) having a capture agent disposed (e.g., bonded) on the nanotriangle. The nanotriangle can be a silver or gold nanotriangle or a silver or gold composite nanotriangle. The nanotriangle can have a longest dimension (e.g., thickness, height, length) of about 50 nm to 2,000 nm and a height of about 10 nm to 100 nm. The silver composite nanotriangle can be a silver-silicon dioxide composite nanotriangle, a silver-magnesium fluoride composite nanotriangle, a silver-titanium dioxide composite nanotriangle, or a silver-metal composite (such as silver (gold)-copper, silver-gold, silver (gold)-aluminum, etc.) or gold-metal composite (such as gold-copper, gold-aluminum, etc.). The substrate can be made of any transparent materials that allow the LSPR signals specified to the size of the nanotriangles to pass through, such as a smooth piece of glass, polydimethylsiloxane (PDMS), polyethylene (PE), polycarbonate (PC), acrylic, and the like. The detecting module can be an array of addressable nanotriangles. In an aspect, the detecting module can include multiple areas (e.g., wells) that each include a plurality of nanotriangles, where the sample fluid can be disposed therein. In addition, a magnetic particle (e.g., a nanoparticle or microparticle) can be used to mix the sample fluid, which can reduce the time for the biological agents to interact with the target agents (e.g., reduce time needed process the sample).

The capture agent has an affinity for the biological agent, where the biological agent can be present in a sample such as a fluid (e.g., saliva, blood, urine). The capture agent can include: a protein, an antibody (monoclonal or polyclonal), an antigen, a polynucleotide, an enzyme, a hapten, a polysaccharide, a sugar, a fatty acid, a steroid, a glycoprotein, a carbohydrate, a lipid, a purine, a pyrimidine, an aptamer, a small molecule, a ligand, or combinations thereof. In an aspect, the capture agent can be ACE-2 protein.

The term “affinity” can include biological interactions and/or chemical interactions. The biological interactions can include, but are not limited to, bonding or hybridization among one or more biological functional groups located on the biological target and/or the capture agent. The chemical interaction can include, but is not limited to, bonding among one or more functional groups (e.g., organic and/or inorganic functional groups) located on the capture agent and/or biological agent. In an aspect, the capture agent has a strong preference (e.g., 90% or more, 95% or more, 99% or more, or 99.9% or more) to bond with the biological agent over other components that might be present in the fluid of the sample so that the capture agent is an effective way to sense and detect the presence of the biological agent in the samples of interest, such as saliva, blood, urine, or other bodily fluids.

The term “bound”, “bond”, or “bonded” can include, but is not limited to, chemically bonded (e.g., covalently or ionically), biologically bonded, biochemically bonded, and/or otherwise associated with the particle. In an embodiment, “bound”, “bond”, or “bonded” can include, but is not limited to, a covalent bond, a non-covalent bond, an ionic bond, a chelated bond, as well as being bound through interactions such as, but not limited to, hydrophobic interactions, hydrophilic interactions, charge-charge interactions, π-stacking interactions, combinations thereof, and like interactions.

In an aspect, “biological agent” is intended to encompass microorganisms such as viruses, bacteria, protozoa, archaea, fungi, algae, spores, apicomplexan, trematodes, nematodes, mycoplasma, or combinations thereof. In addition, the biological target can include native intact cells, viruses, bacterium, and the like. The biological agent can include a coronavirus such as SARS-COV-2 and its variants.

In an example, the sample can include about 10 or 20 μL of patient body fluid samples, for example from saliva or nasal swap through dilution of buffer. In aspect the sample can be a nasopharyngeal swab samples (NPAs) are the standard used in CoV2 and other virus testing. NPAs have been used to show very high CoV2 during the first week of symptoms with a peak at 7.11×10⁸ RNA copies per throat swab on day 4. Infectious virus was readily isolated from samples derived from the throat or lung. (See, DOI https://doi.org/10.1038/s41586-020-2196-x). The liquid and the substrate (e.g., the nanotriangles having the capture agent) interact for less than 20 minutes, then the well containing the nanotriangle can be washed three times with de-ionized water or molecular water. To shorten the interaction time, the 10 μL body fluid sample can mixed with 10 μL of magnetic nanoparticle suspension (e.g., having a longest diameter of about 20 nm to 100 nm, concentration 0.01 mg/mL to 10 mg/mL), and placed in a rotating magnetic field (e.g., field magnitude from 1 mT to 100 mT, and frequency from 10 Hz to 500 Hz) for a desired amount of time (e.g., about 15 min or less, about 10 min or less, about 5 min or less, etc). After the desired time, the well will be washed three times before performing UV-Vis-NIR spectrum measurement. After the interaction and washing, the UV-Vis-NIR transmission spectrum of the nanotriangles will be taken and compared to the previously (without the patient sample) taken transmission spectrum to determine whether there are virus presented in the patient sample and roughly the amount of virus in the sample using a pre-calibrated curve.

The present disclosure also provides for systems for detecting a biological agent, where the system includes the white light source, the UV-Vis-NIR spectrometer, and the detecting module, such as described herein. Prior to the introduction of sample of fluid to the detecting module, the UV-Vis-NIR spectrometer is configured to detect a first localized surface plasmon resonance (LSPR) signal that corresponds to the nanotriangle having the capture agent. After introduction of the sample of fluid that contains the biological agent, the UV-Vis-NIR spectrometer is configured to detect a second LSPR signal that corresponds to the nanotriangle having the capture agent and the biological agent bonded to the capture agent. When the first LSPR signal and the second LSPR signal are different, the difference corresponds to the amount of biological agent present in the sample. When the first LSPR signal and the second LSPR signal are the same, the biological agent is not present in the sample. The system is configured to detect the presence of the biological agent within 20, 15, 10, or 5 minutes or less upon introduction of a sample to the detecting module.

In an aspect, the present disclosure includes method of detecting a biological agent. The method can include, among other steps, disposing a sample of a fluid onto an area of the detecting module as described hereon. Prior to disposing the sample, the detecting module includes a capture agent bonded to the nanotriangle and a first localized surface plasmon resonance (LSPR) signal is known that corresponds to the nanotriangle bonded to the capture agent. For example, a standard can be prepared that includes the baseline signal without the biological agent bonded to the capture agent and/or a calibration curve to add precision to the determine of the amount of biological agent present in the sample. Optionally, once the sample is disposed with the nanotriangles, the sample can be mixed with a magnetic particle. Subsequently, a second LSPR signal can be detected using the UV-Vis-NIR spectrometer after disposing the sample in the first area. When the second LSPR signal is different than the first LSPR signal, this indicates that the biological agent is bonded to the capture agent, which indicates that the biological agent is present in the sample. The magnitude of the difference between the first LSPR signal and the second LSPR signal corresponds to the amount of biological agent present in the sample. When the first LSPR signal and the second LSPR signal are the same the biological agent is not present. The method of detecting the presence of the biological agent within 20, 15, 10, or 5 minutes or less upon introduction of a sample to the detecting module.

In another aspect, the present disclosure includes methods of making a detecting module, such as those described herein. A substrate having an array of addressable nanotriangles is provided (See for example, Analyst, 2020, 145, 7654-7661 regarding making then nanotriangles, which is incorporated herein by reference in regard to making the nanotriangles) and then a capture agent is introduced to the substrate including the array of addressable nanotriangles, where the capture agent bonds to the nanotriangles. The capture agents not bonded to the addressable nanotriangles are removed (e.g., rinsed, washed). In order to prevent unintentional bonding of components of the sample with nanotriangles having no a biological agent, a second agent (e.g., BSA) is introduced to the substrate to bond to the nanotriangles that do not already have a capture agent bonded to it. Then the unbound second agents can be removed (e.g., rinsed, washed).

The present disclosure provides for the following embodiments.

The present disclosure provides for a device for detecting a biological agent, comprising: a white light source; a UV-Vis-NIR spectrometer; and a detecting module comprising a substrate including a plurality of nanoparticles (e.g., nanotriangles) having a capture agent disposed on the nanoparticles, wherein the capture agent has an affinity for a biological agent, wherein the biological agent is a type of virus or a type of bacteria, wherein the white light source, the UV-Vis-NIR spectrometer, and the detecting module are interfaced so that the light from the white light source is configured to be directed towards the substrate of the detecting module, wherein the UV-Vis-NIR spectrometer is configured relative to the detecting module to receive a signal from the nanoparticles as a result of the light from the white light source interacting with the nanoparticles. In an aspect, the type of virus is a coronavirus. In an aspect, the coronavirus is SARS-COV-2 or variant thereof. In an aspect, the nanoparticle is a nanotriangle such as a silver nanotriangle, a silver composite nanotriangle, a gold nanotriangle, or a gold composite nanotriangle. In an aspect, the nanotriangle has a longest dimension of about 50 nm to 2000 nm and a height of about 10 nm to 100 nm. In an aspect, the silver composite nanotriangle or gold composite nanotriangle is a silver-silicon dioxide composite nanotriangle, a silver-magnesium fluoride composite nanotriangle, a silver-titanium dioxide composite nanotriangle, silver-copper composite nanotriangle, silver-gold composite nanotriangle, silver (gold)-aluminum composite nanotriangle, silver-copper composite nanotriangle, or gold-aluminum composite nanotriangle. In an aspect, the capture agent is an angiotensin-converting enzyme 2 (ACE-2) protein. In an aspect, the capture agent is an enzyme, aptamer, or hapten. In an aspect, the device is configured to detect the presence of a biological agent within 20 minutes upon introduction of a sample to the detecting module, wherein the sample includes the biological agent. In an aspect, the substrate is an array of the plurality of nanotriangles at addressable locations. In an aspect, the UV-Vis-NIR spectrometer is configured to detect a change in the localized surface plasmon resonance (LSPR) of the nanotriangle with the capture agent bound to the nanotriangle and when the biological agent is bonded to the capture agent that is bonded to the nanotriangle.

The present disclosure provides for a method of detecting a biological agent, wherein the method includes: disposing a sample of a fluid onto an area of the detecting module as provided above and herein, wherein prior to disposing the sample, the detecting module includes a capture agent bonded to the nanotriangle and wherein a first localized surface plasmon resonance (LSPR) signal is known that corresponds to the nanotriangle bonded to the capture agent; and detecting a second LSPR signal using the UV-Vis-NIR spectrometer after disposing the sample in the first area, wherein when the second LSPR signal is different than the first LSPR signal, this indicates that the biological agent is bonded to the capture agent, which indicates that the biological agent is present in the sample. In an aspect, first LSPR signal and the second LSPR signal are the same if the biological agent is not present. In an aspect, the magnitude of the difference between the first LSPR signal and the second LSPR signal corresponds to the amount of biological agent present in the sample. In an aspect, the method of detecting takes 20 minutes or less. In an aspect, prior to detecting, the sample in the area is mixed. In an aspect, the mixing is performed using a magnetic nanoparticle. In an aspect, the method of detecting takes 10 minutes or less.

The present disclosure provides for a system for detecting a biological agent, comprising: a white light source; a UV-Vis-NIR spectrometer; and a detecting module, wherein the white light source is configured to direct a white light at the detecting module, wherein the UV-Vis-NIR spectrometer is configured to detect a signal corresponding to nanotriangle present in the detecting module; wherein the detecting module includes a substrate including a plurality of the nanoparticles (e.g., nanotriangles) having a capture agent disposed on the nanoparticle, wherein the capture agent has an affinity for a biological agent, wherein the biological agent is a type of virus or a type of bacteria, wherein prior to the introduction of sample of fluid to the detecting module, the UV-Vis-NIR spectrometer is configured to detect a first localized surface plasmon resonance (LSPR) signal that corresponds to the nanoparticle having the capture agent, wherein after introduction of the sample of fluid that contains the biological agent, the UV-Vis-NIR spectrometer is configured to detect a second LSPR signal that corresponds to the nanoparticle having the capture agent and the biological agent bonded to the capture agent, wherein the first LSPR signal and the second LSPR signal are different, wherein the difference corresponds to the amount of biological agent present in the sample. In an aspect, when the first LSPR signal and the second LSPR signal are the same, the biological agent is not present in the sample. In an aspect, the system is configured to detect the presence of the biological agent is about 20 minutes or less from the time the sample is introduced to the detecting module. In an aspect, the detecting module is configured to mix the sample. In an aspect, the detecting module is configured to mix the sample using a magnetic nanoparticle. In an aspect, the system is configured to detect the presence of the biological agent is about 10 minutes or less from the time the sample is introduced to the detecting module. In an aspect, the nanotriangle is a silver nanotriangle, a silver composite nanotriangle, a gold nanotriangle, or a gold composite nanotriangle.

The present disclosure provides for a method of making a detecting module, comprising: providing a substrate having an array of addressable nanotriangles, introducing a capture agent to the substrate including the array of addressable nanotriangles, wherein the capture agent bonds to the nanotriangles, removing the captures agents not bonded to the addressable nanotriangles, introducing a second agent to the substrate including the addressable nanotriangles, wherein the addressable nanotriangles that do not have a capture agent bonded to it will bond with the second agent; and removing the second agents not bound to the addressable nanotriangles. In an aspect, the nanotriangle is a silver nanotriangle, a silver composite nanotriangle, a gold nanotriangle, or a gold composite nanotriangle. In an aspect, the nanotriangle has a longest dimension of about 50 nm to 2000 nm and a height of about 10 nm to 100 nm. In an aspect, the silver composite nanotriangle or gold composite nanotriangle is a silver-silicon dioxide composite nanotriangle, a silver-magnesium fluoride composite nanotriangle, a silver-titanium dioxide composite nanotriangle, a silver-copper composite nanotriangle, a silver-gold composite nanotriangle, a silver (gold)-aluminum composite nanotriangle, a silver-copper composite nanotriangle, or a gold-aluminum composite nanotriangle. In an aspect, the capture agent is an angiotensin-converting enzyme 2 (ACE-2) protein. In an aspect, the capture agent is an enzyme, aptamer, or hapten.

Now having described embodiments of the present disclosure, additional details are provided in Example 1.

### Example 1

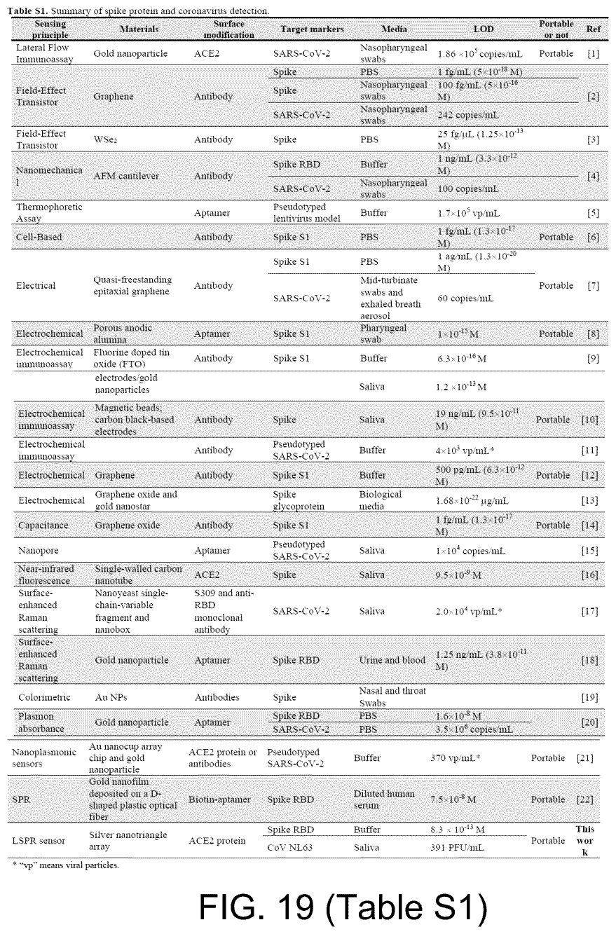

The SARS-COV-2 pandemic COVID-19 disease has led to unprecedented burden on national and international healthcare. This has motivated researchers to develop reliable tools to aid SARS-CoV-2 diagnostics. The current molecular diagnostic tests for SARS-COV-2 can be classified into two categories, i.e., nucleic acid tests and serological/immunological tests. The identification of SARS-CoV-2 typically involves viral RNA based reverse transcriptase real-time polymerase chain reaction (RT-PCR) and nucleic acid hybridization strategies [1, 2]. RT-PCR is the ‘gold standard’ and has excellent selectivity and sensitivity and is laboratory-based [3], but this detection requires viral RNA extraction and expertise in PCR which is time-consuming and requires qualified personnel. Similarly, immunological tests may take days-to-weeks after the onset of symptoms for a patient to develop a detectable antibody level [4]. Although the IgM/IgG rapid test kits are available, false-positive results may occur [5]. Recently, direct detection of SARS-COV-2 using reverse transcription loop-mediated isothermal amplification (RT-LAMP) on heat-inactivated samples has become available [6]. However, the development of rapid and highly accurate biosensors for coronavirus (CoV) is still needed. Table S1 (FIG. 19) in the Supplementary Materials (SM) summarizes some of the recent development and the corresponding sensing performance. Among different proposed methods, localized surface plasmon resonance (LSPR)-based optical sensors are one of the potential entrants as a rapid SARS-CoV-2 sensor, have attracted notable attention and been widely studied over the past decade [7-9]. The resonance absorbance wavelength of the LSPR sensor responds to changes in the local dielectric environment. These sensors are compact, durable, repeatable, and more reliable than traditional sensors, offering real-time and label-free chemical and biological detection. Label-free LSPR biosensors for SARS-COV-2 screening have been reported for nucleic acids [10, 11], surface protein subunits (spike, envelope, and membrane) [12], and SARS-COV-2 virus [13, 14], as well as antibodies (IgG, IgM) [15-17]. For example, Ventura et al. proposed a colorimetric sensor using gold nanoparticles for SARS-COV-2 surface spike protein detection [12]. Huang et al. developed a double-antibody sandwich plasmonic resonance immunoassay for SARS-COV-2 pseudovirus detection using Au nanocup array chip and gold nanoparticles, and reported a LOD of 370 virus particle/mL [14]. But this sandwich detection strategy and sensor preparations are complicated. Very recently, an aptamer-functionalized gold nanoparticle based sensor was reported and was able to detect 16 nM spike protein and 3.54×10⁶ genome copies/mL of inactivated SARS-COV-2 [13]. However, sliver should have a higher plasmonic effect compared to Au [18]. And the rapid and cost-effective LSPR sensor chips still need to be developed for direct detections of SARS-COV-2 or coronavirus (CoV).

In this Example, a human angiotensin-converting enzyme 2 protein (ACE2) functionalized silver nanotriangle (AgNT) array LSPR sensor is developed with high sensitivity and specificity of SARS-COV-2 spike RBD proteins and CoV NL63. Linear relationship can be observed both from the transmission intensity at 750 nm and the redshift of the LSPR wavelength versus the logarithm of spike RBD protein concentration in the concentration ranging from 0.38 pM to 9420 pM. For NL63, the detection range extended from 625 to 10⁴ PFU/mL in untreated saliva. The detection time is determined to be less than 20 min. This rapid optical sensor can be expanded as a potential point-of-care COVID-19 diagnostic platform for real sample analysis.

### Materials and Methods

### Materials:

Polystyrene nanospheres (PSNS) with 500 nm diameter (Polyscience, Lot #679675) were used to form the colloid monolayer onto clean glass slides (Gold Seal, Part #301). Sulfuric acid (Fisher Scientific, 98%), ammonium hydroxide (Fisher Scientific, 98%), and hydrogen peroxide (Fisher Scientific, 30%) were acquired to clean the glass slides. Silver (Kurt J. Lesker, 99.999%) and titanium pellets (Kurt J. Lesker, 99.995%) were purchased as the evaporation materials. Methanol, acetone, 1-hexanol (Tokyo Chemical Industry Co., >98%), chloroform (J. T. Baker, 99%), tetrachloromethane (Sigma-Aldrich, 99.9%), and toluene (Fisher Scientific, 99.8%) were used to characterize the refractive index (RI) sensing performance of the AgNT array. Human angiotensin-converting enzyme 2 protein (Sino Biological) and SARS-COV-2 spike receptor binding domain protein (spike RBD; Sino Biological) were purchased for surface functionalization and sensing application. Bovine serum albumin (Sigma) was purchased for surface blocking. Sodium phosphate dibasic (Na₂HPO₄; ≥99.0%), sodium phosphate monobasic (NaH₂PO₄; ≥99.0%), sodium chloride (NaCl; ≥99.0%), trehalose, mannitol, and Tween-80 were purchased from Sigma. PBS buffer (100 mM Na₂HPO₄, 100 mM NaH₂PO₄, 300 mM NaCl, pH=7.4), phosphate buffer (100 mM Na₂HPO₄ and 100 mM NaH₂PO₄, pH=7.4) and dilution buffer (1 mL PBS containing 0.05 g trehalose, 0.05 g mannitol, 0.1 μL Tween-80) were prepared and used for the treatment of proteins. Dulbecco's Modified Eagles Medium (DMEM; GIBCO BRL laboratories, Grand Island, NY) supplemented with 1% fetal bovine serum (FBS; Hyclone Laboratories, Salt Lake City, UT) was used as cell culture media. Deionized (DI) water (18 MΩ) was used throughout all the experiments. All chemicals and materials were used without further purification.

### Sensing Strategy:

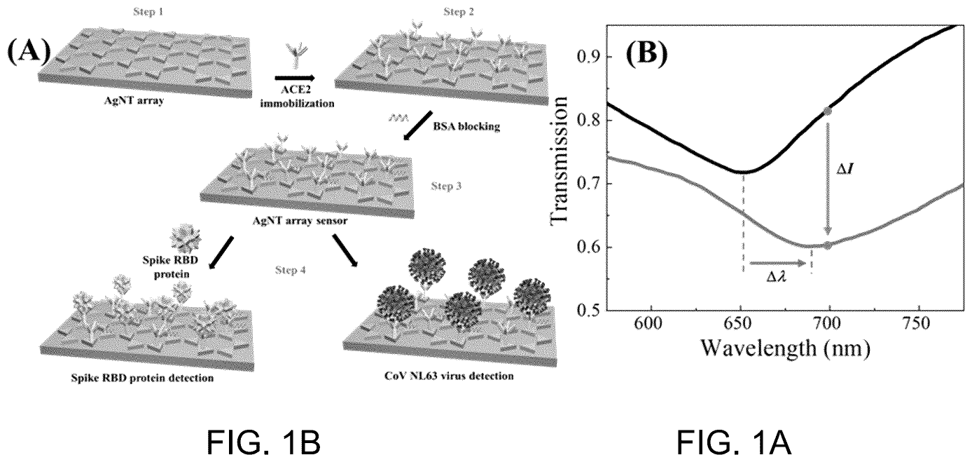

The general strategy of LSPR sensor for spike RBD and coronavirus detection is illustrated in FIG. 1A. It consists of four steps: Step 1: AgNT array fabrication. Step 2: ACE2 immobilization. Step 3: BSA blocking. Step 4: spike RBD protein or virus detection. The AgNT array is immobilized with the ACE2 proteins via electrostatic and hydrophobic interactions. During the detection, the ACE2 proteins on AgNT can specifically capture desired coronaviruses or spike RBD. Such a binding can change the optical property of the AgNT as shown in FIG. 1B: it is expected that before detection, the transmission spectrum T(λ) of the AgNT shows a dip (the black curve); after binding of desired analyte, the T(λ) changes to the red curve, the dip location redshifts (green arrow) and the transmission amplitude decreases (blue arrow). These changes signify a positive detection.

### Fabrication of AgNT Array:

The general fabrication procedure is outlined in FIG. 7A. Prior to PSNS monolayer formation, the glass substrates (2.54 cm×0.9 cm) were cleaned with sulfuric acid, ammonium hydroxide, and hydrogen peroxide. All substrates were then rinsed in deionized water. The uniform PSNS (diameter 500 nm) monolayers with large monocrystalline domains were prepared on glass substrates via an air-water interface method [19]. The PSNS monolayer-coated substrates were loaded into a custom-designed electron beam deposition system with the substrate normal antiparallel to the incident vapor direction. The vacuum chamber was pumped down under a base pressure of <10⁻⁶ Torr. A 3 nm layer of Ti was deposited at a rate of 0.2 nm/s followed by a 60 nm Ag film deposited at 0.3 nm/s under a high vacuum condition. The deposition rate and vapor thickness on the substrate were monitored by a quartz crystal microbalance. After the Ag deposition, the monolayer template was removed using Scotch tape, and the substrates were rinsed in toluene, acetone, and 2-propanol to remove PSNS residue. Subsequently, a PDMS layer with arrayed small wells (3 wells, with the well diameter of 4 mm, well depth of 1 mm) was molded on the AgNT array to restrict the effective sensing areas (FIG. 7B), and we refer them as AgNT wells.

### Immobilization of ACE2 on AgNT Array:

The ACE2 protein was immobilized on the AgNT array via electrostatic and hydrophobic interactions. First, 200 μL ACE2 protein at 0.62 mg/mL was dialyzed in phosphate buffer (100 mM Na₂HPO₄ and 100 mM NaH₂PO₄, pH=7.4). The dialyzed ACE2 solution was diluted to 66 μg/mL with phosphate buffer. 20 μL ACE2 solution was transferred into each AgNT well and incubated for 2 h at room temperature. Then, the wells were washed with DI water 3 times. Subsequently, 20 μL of 1 mg/mL BSA solution was transferred to a AgNT well and incubated for 2 h in order to block the ACE2 uncovered area of AgNT and avoid nonspecific binding of spike RBD protein or virus particles. Subsequently, the wells were rinsed with DI water and air-dried. The ACE2 protein modified AgNT array substrates (AgNT sensors) were now ready for the detection. The corresponding optical transmission spectra of the same AgNT well was measured after each step of surface modification.

### Spike RBD Protein Detection:

20 μL spike RBD protein ranging from 2.03 pM to 9420 pM in dilution buffer were transferred into different ACE2 functionalized AgNT wells and incubated for 2 h at room temperature. Then, the AgNT array sensors were washed with DI water and air-dried for optical transmission measurements.

### Virus Incubation:

All the virus experiments, including virus preparation, characterization, and LSPR measurements, were conducted in a biosafety level 2 (BSL-2) environment. CoV NL63, CoV 229E, and CoV OC43 were propagated in Vero E6 cells which were maintained in DMEM supplemented with 1% heat-inactivated (56° C.) FBS. Briefly, cells were infected using a multiplicity of infection (MOI)=0.1. After 48 h, the viruses were harvested in serum-free DMEM followed by two freeze-thaws (−70° C./4° C.), after which the contents were collected and centrifuged at 4000 g for 15 min at 4° C. The virus titers were similar, i.e., 10⁵ PFU/mL, determined by immunostaining plaque assay as previously described [20]. Table S2 lists the different kinds of coronavirus as well as corresponding receptors. There are three kinds of coronavirus, CoV NL63, SARS-COV, SARS-COV-2, which are specific to ACE2 protein. The experiments on SARS-COV and SARS-COV-2 must be conducted in BSL-3 lab, while CoV OC43, CoV 229E, and CoV NL63 can be handled in BSL-2 environment.

### Coronavirus (CoV) Detection:

CoV NL63, CoV 229E or CoV OC43 cell-free supernatant suspensions ranging from 391 to 10⁵ PFU/mL in PBS buffer were transferred into different ACE2 functionalized AgNT wells and incubated for 2 h at room temperature. Subsequently the AgNT wells were washed 3× with DI water and air-dried for optical transmission measurements. Virus spiked saliva samples were prepared by adding different concentrations of CoV NL63 to saliva to achieve final concentrations ranging from 625 to 10⁴ PFU/mL for detection. To mimic the non-pretreated saliva sample, saliva samples for each AgNT well were prepared by adding 2 μL of different concentrations of coronavirus solutions to 18 μL of saliva, so the original coronavirus solutions were diluted 10 folds, i.e., the maximum viral concentration in the saliva sample for detection was only 1×10⁴ PFU/mL.

### Instruments:

The morphology of the AgNT array was characterized by atomic force microscopy (AFM, Park Systems NX-10 AFM). The optical transmission spectra of the as deposited AgNT arrays were measured by an ultraviolet-visible spectrophotometer (UV-Vis, Jasco-750). For virus detection in a BSL-2 environment, a portable UV-Vis spectrophotometer was used for transmission measurement of CoV detection in a biosafety level-2 hood and a pair of pinholes were added between the incident light and sample surface to restrict the incident light to a smaller area. The setup also consisted of a halogen source (Tungsten Halogen HL-2000), a spectrophotometer (Ocean Optics USB2000) and two optical fibers. The transmission spectra Tₛ(λ) and Tᵣ(λ) of AgNT wells and reference sample (bare glass slide) were measured separately, and the final transmission spectra T(λ) was calculated as

$$T ( λ ) = {T}_{s} ( λ ) / {T}_{r} ( λ ) .$$

### Results and Discussion:

### Characterization of AgNT Array:

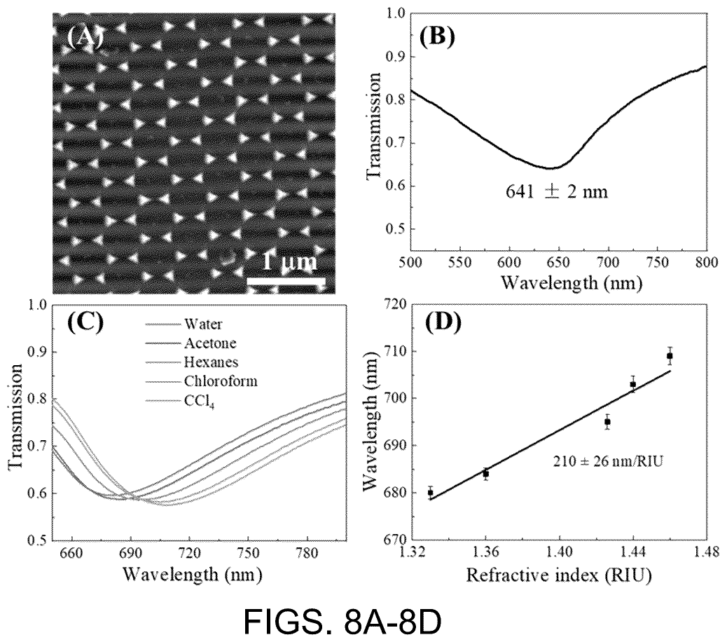

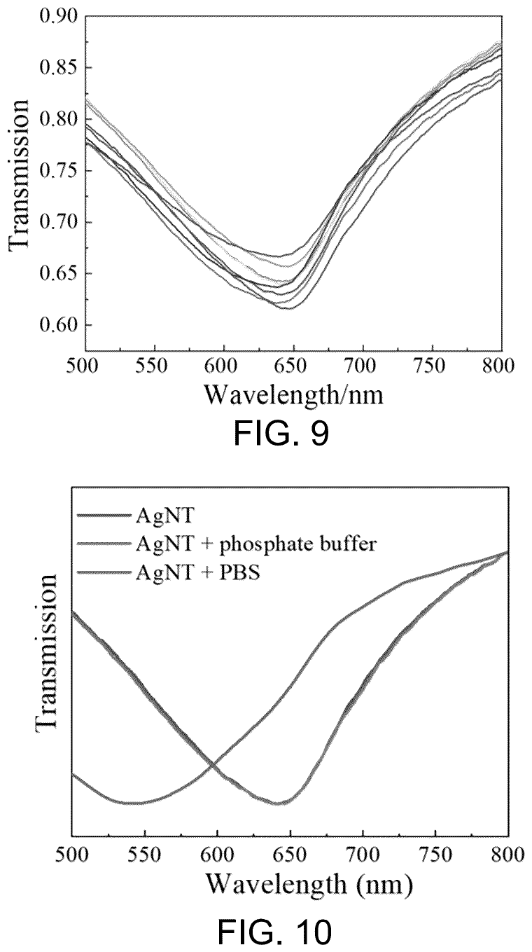

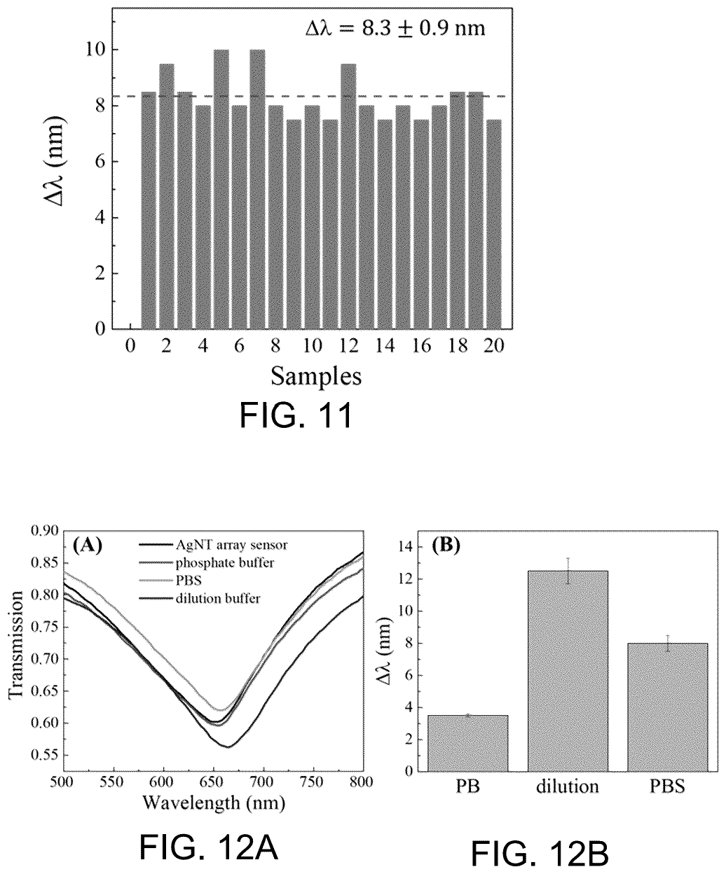

FIG. 8A shows a representative AFM image of AgNT array fabricated via the 500 nm PSNS monolayer. As expected, equilateral nanotriangle arrays with the side length of 152±3 nm are formed, and the height of each triangle is 60±1 nm. A distinct dip do in UV-Vis transmission spectrum (FIG. 8B) is found at λ₀=641±2 nm, taking from 8 different AgNT samples (FIG. 93). The refractive index (RI) sensitivity of the AgNT array is evaluated by measuring the λ₀ shift when the AgNT array is immersed in solutions with different refractive indices, and the corresponding transmission spectra are shown in FIG. 8C. The change of λ₀ versus refractive index (RI) is plotted in FIG. 8D, and the slope gives the sensitivity of the LSPR sensor, S=210±10 nm/RIU, where RIU refers to refractive index unit. A similar sensitivity of 191 nm/RIU has been reported for a similar structure [21]. Even though this AgNT array does not have a high RI sensitivity, the AgNT array is straightforward to fabricate and has large uniform area. The nanofabrication procedure for AgNT array is relatively inexpensive compared to gold nanostructures or other nanostructures fabricated either by focus ion beam method or by electron beam lithography method.

### Optimize ACE2 Protein Immobilization on the AgNT Array:

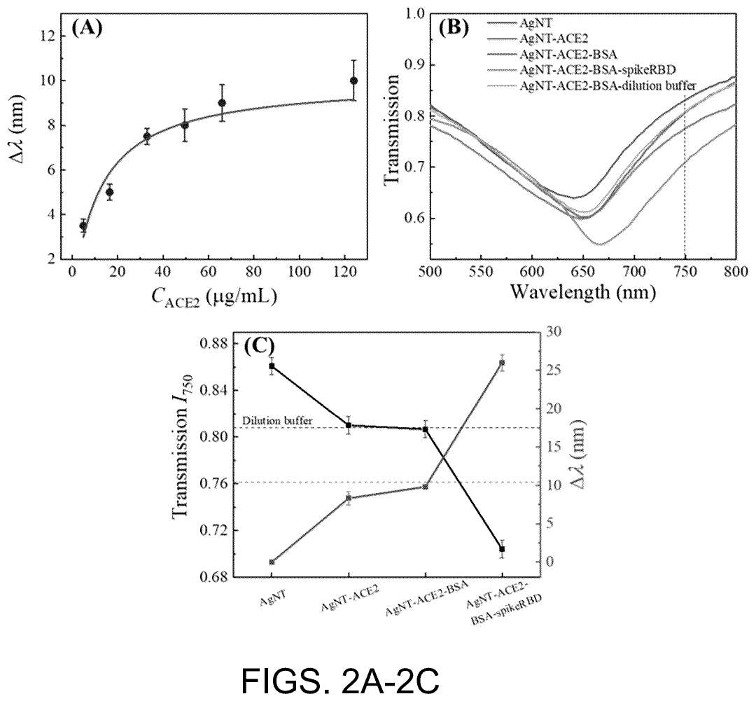

ACE2 is the cellular receptor for NL63, SARS-COV, and SARS-COV-2 [22]. Previous studies have demonstrated that the S1 domains of coronaviruses contain the receptor-binding domains (RBDs) that directly bind to the cellular receptors [23]. To achieve specific detection of spike RBD protein and NL63, ACE2 protein is used to functionalize the AgNT array. However, Ag is not chemically stable in NaCl solution [24], rather is found to be stable in phosphate buffer (see Section S1 in SM). Thus, phosphate buffer was used to dialyze the ACE2 solution to remove the chloride ions and for further ACE2 immobilization. To optimize the ACE2 protein immobilization on the AgNT array, 20 μL ACE2 solutions of a series of concentrations ranging from 4.96 to 124 μg/mL were transferred into different AgNT wells. Transmission spectra of AgNT wells were measured before and after the ACE2 immobilization. The redshift of λ₀, i.e., Δλ=λ_{ACE2}−λ₀, versus different concentration C_{ACE2} of ACE2 protein is plotted in FIG. 2A. The Δλ initially increases rapidly with C_{ACE2} when C_{ACE2} is small; then approaches to a constant when C_{ACE2}>40 μg/mL. This relationship follows the Langmuir isotherm adsorption model for proteins [25],

$$Δ λ = Δ {λ}_{max} ( \frac{{kC}_{ACE 2}}{1 + {kC}_{ACE 2}} ) , \qquad ( 1 )$$

where Δλₘₐₓ is the maximum LSPR shift measured when the ACE2 protein is fully covering the AgNT and k is the surface binding constant of the ACE2 to Ag surface. The solid curve in FIG. 2A shows the best fitting with k=0.08±0.03 mL/μg and Δλₘₐₓ=10±1 nm. The adsorption of ACE2 protein on the silver surface is mainly dependent on electrostatic interaction and the Ag—S covalent bond [26]. ACE2 proteins can bind to silver surfaces through either free amine groups or cysteine residues in the proteins and via the electrostatic attraction of negatively charged carboxylate groups. Those amino acids with residues containing sulfur atoms can form covalent bonds with silver. The binding constant is influenced by these factors as well as the surface coverage of the Ag film. According to the fitting, for C_{ACE2}>66 μg/mL, the Δλ (=8.3 nm at C_{ACE2}=66 μg/mL) does not change too much. Therefore, C_{ACE2}=66 μg/mL of ACE2 has been selected as the optimized immobilization concentration. The success of the ACE2 immobilization on Ag surface was further confirmed by the surface enhance Raman scattering (SERS) spectroscopy as shown in Section S2 of SM.

The general sensing strategy consists of four steps: Step 1: AgNT array fabrication; Step 2: ACE2 coating; Step 3: BSA blocking; Step 4: spike RBD protein detection. The corresponding transmission spectra for each step are shown in FIG. 2B and the changes of the transmission intensity I₇₅₀ and LSPR wavelength Δλ are shown in FIG. 2C. When AgNT array is incubated with 66 μg/mL ACE2 protein, λ₀ red shifts 8.3±0.9 nm, I₇₅₀ decreases from 0.86±0.01 to 0.810±0.007, indicating that ACE2 proteins have been immobilized on the AgNT array. After BSA blocking, λ₀ further red shifts 1.2 nm, and I₇₅₀ decreases to 0.807±0.008. Such a small change in I₇₅₀ indicates that the BSA only blocks a small fraction of the AgNT surface area. When adding spike RBD protein of a concentration C_{spikeRBD}=9420 pM, λ₀ has a huge jump, increasing 16±1 nm, while I₇₅₀ also significantly reduces to 0.704±0.008, indicating that the spike RBD proteins bind to ACE2 proteins. Compared both the changes of Δλ and I₇₅₀ between adding spike RBD protein and BSA blocking, this AgNT array sensor does show good specificity for spike RBD protein detection. When only adding dilution buffer without spike RBD protein, both Δλ and I₇₅₀ are found to be around 10.2 nm and 0.808 (the dashed lines in FIG. 2C), which is within the accuracy of the detection system. Also, with only the PBS buffer, the LSPR wavelength Δλ does not blue shift, which demonstrates that ACE2 and BSA protein can protect the silver surface from degradation in the biological environment. So, the functionalized AgNT array sensor is reliable for biosensing applications.

### Spike RBD Protein Detection:

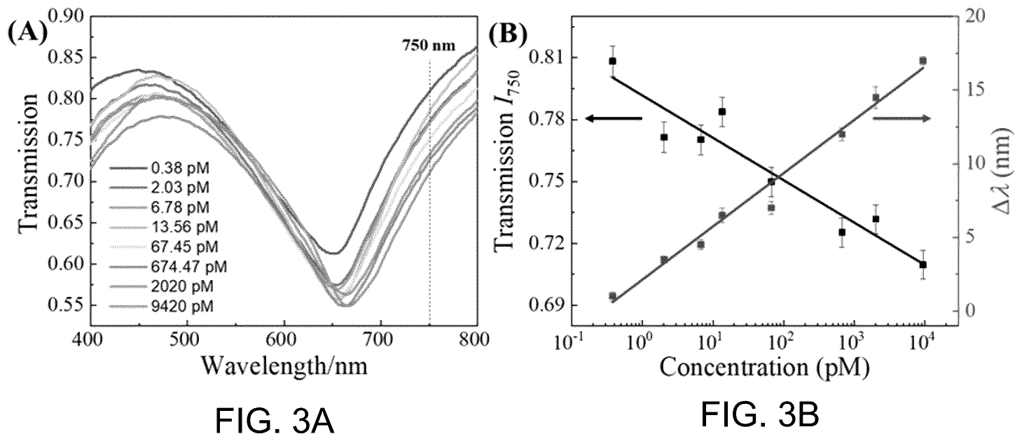

The sensor's responses to spike RBD protein concentration C_{spikeRBD} have been investigated to establish the calibration curve. FIG. 3A shows the original UV-Vis spectra, 1750 decreases while λ₀ increase monotonically with C_{spikeRBD}. In a semi-log plot as shown in FIG. 3B, both I₇₅₀ and Δλ are found following a linear relationship with C_{spikeRBD} in the region from 0.38 pM to 9420 pM. For I₇₅₀−C_{spikeRBD}, the best fitting gives I_{spikeRBD}=−0.021×log[C_{spikeRBD}]+0.792, where the goodness of fitting R² is 0.915, whereas for Δλ−C_{spikeRBD}, Δλ follows Δλ_{spikeRBD}=3.6×log [C_{spikeRBD}]+2.1 (nm) with a better R²=0.987. The LOD of spike RBD protein is estimated to be 0.38 pM. Defining the limit of detection (LOD) as the lowest detected concentration whose signal is higher than the blank control signal plus three standard deviations [27, 28], the LOD of spike RBD protein is estimated to be 0.83 pM. This is comparable with those obtained by other methods, including commercial ELISA kits, that usually fall in the pM concentration range [29]. In addition, the total detectable range spans in 4 orders of magnitude, from ˜1 pM to 10⁴ pM, and the actual amount of spike RBD molecules in the detection light beam area is estimated to be from 3×10⁴ to 2×10⁹ molecules shown in Table S4.

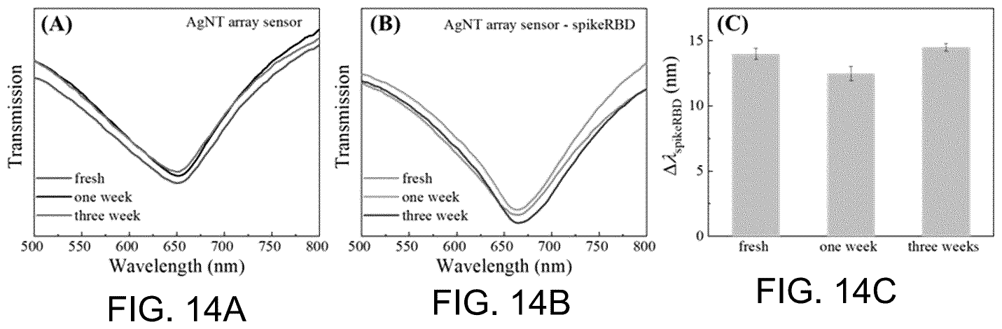

The stability of the AgNT array sensor has also been examined. ACE2 immobilized AgNT array sensors were stored in sealed opaque pouches filled with argon gas and kept at 20° C. for three weeks, then the optical transmission spectra of the ACE2 modified AgNT samples (FIG. 14A) as well as treated by spike RBD protein (C_{spikeRBD}=2020 pM, FIG. 14B) were measured. As shown in FIG. 14A, there is almost no change in λ₀ in AgNT samples after stored for one and three weeks, compared to that of the freshly prepared AgNT samples. Similar result is found for the samples incubating with spike RBD protein solutions (FIG. 14B), and the corresponding redshifts Δλ were found to be 14.0±0.5 nm, 12.5±0.6 nm, and 14.5±0.3 nm, respectively (FIG. 14C), which are comparable to the result reported in FIG. 3B.

### Specificity of AgNT Array Sensor for CoVs:

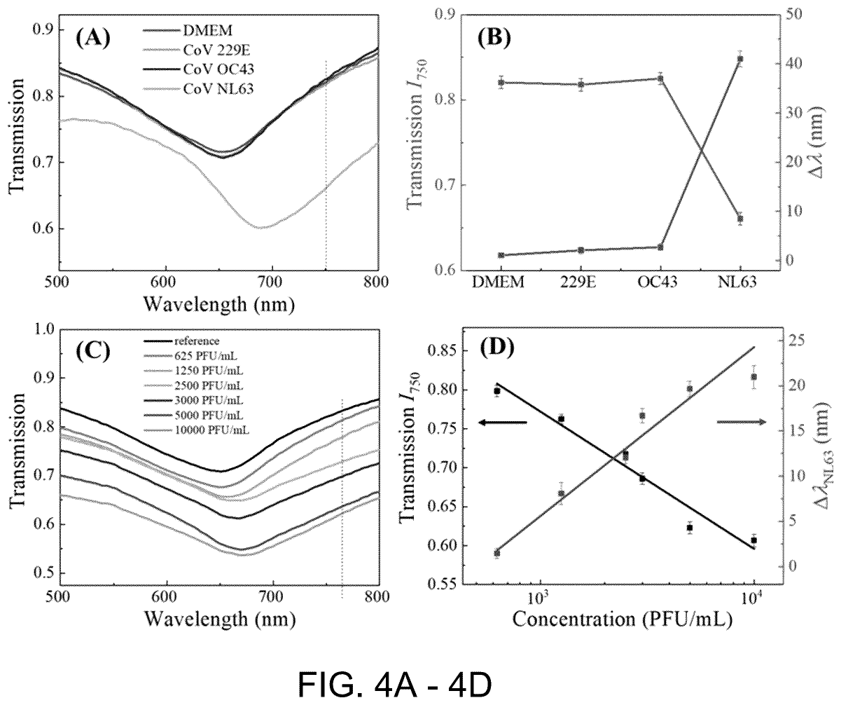

To test the specificity of AgNT array sensor, similar detection experiments have been performed on the three coronavirus strains, i.e., 229E, OC43, and NL63 at the same concentration of 10⁵ PFU/mL in DMEM with 1% FBS. ACE2 is the cellular receptor for NL63, SARS-COV, and SARS-COV-2 [22], which can directly and specifically bind to the S1 domains of the spike protein on these coronaviruses [23]. However, both CoV OC43 and 229E cannot be captured by ACE2 protein. FIG. 4A shows the corresponding transmission spectra and the corresponding changes in I₇₅₀ and Δλ are plotted in FIG. 4B. The values of I₇₅₀ for DMEM buffer, 229E, and OC43 detection all remain around 0.820. However, for NL63 detection at the concentration of 10⁵ PFU/mL, the value of I₇₅₀ decreases significantly to around 0.665. In addition, Δλ caused by DMEM buffer (with 1% FBS) is around 1 nm, and the average Δλ for 229E and OC43 is around 2 nm, which is significantly lower compared to Δλ=40 nm for NL63. These results demonstrate that the ACE2 functionalized AgNT array sensor has good specificity to CoV NL63.

### Specific Detection of NL63 in PBS and in Untreated Saliva:

The practicability of the AgNT array sensor was verified by detecting CoV NL63 in untreated saliva with different concentrations, ranging from 625 to 10⁴ PFU/mL. Saliva without adding NL63 was used as the reference sample. FIG. 4C shows the concentration-dependent transmission spectra of NL63 detection, indicating that the value of I₇₅₀ decreases continuously with NL 63 concentration and the LSPR wavelength λ₀ redshifts monotonically. As shown in FIG. 4D, the semi-log concentration dependent I₇₅₀ plot demonstrates a linear relationship, which can be written as I₇₅₀=−0.18×log [C_{NL63}]+1.30 with R²=0.959. The semi-log concentration dependent Δλ_{NL63} plot also shows a linear relationship, which follows Δλ_{NL63}=19×log [C_{NL63}]−50 with R²=0.965. The LOD is 625 PFU/mL. These results indicate that the AgNT array sensor has good performance in complex biological environments. For the LOD, as shown in Table S1 for different sensors, the LODs are given in different units and there is no definite relationship between different units. For example, PFU/mL, copies of RNA, or number of viral particles/mL. In general, one tends to assume that one PFU corresponds to one viral particle and one viral particle only contains one RNA [30, 31]. However, there are exceptions, for example, the PFU/viral particle ratio for varicella-zoster virus is 40,000:1 and the RNA copies to viral particle varies for different variants of CoV-SARS-2 viruses [33]. In order to have a systematic comparison, a side-by-side PCR measurements and LSPR measurements should be conducted in order to obtain a more qualitative relationship.

### Estimation of the Number of Detected Viruses on AgNT:

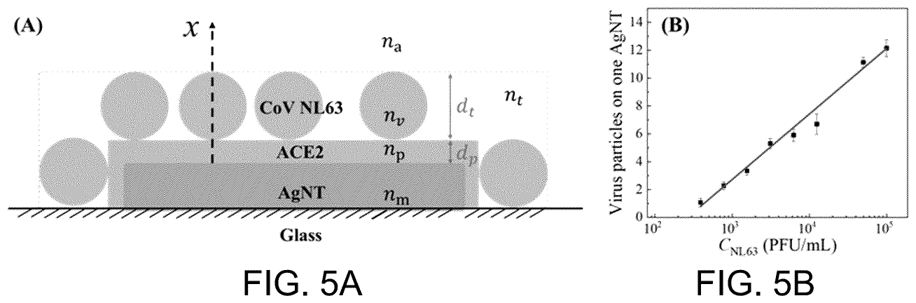

Based on the detection strategy shown in FIG. 1, the whole detection process can be considered as coating the AgNT with two dielectric layers as shown in FIG. 5A. The first coated layer is a uniform layer of ACE2 protein with an RI nₚ=1.45 [34, 35]. BSA blocking can be ignored or included in the first layer, because the Δλ of BSA blocking is only 1.5 nm. The second layer is NL63 with refractive index of each particle nₚ=1.8 [36]. According to Li et al., the electric field in the surrounding layers decays exponentially from the surface of AgNT with a characteristic decay length δ[37]. Therefore, the effective refractive index of each coated layer is integrated by the local refractive index from zero to infinity [21, 38],

$${n}_{eff} = \frac{2}{δ} {\int }_{0}^{∞} n ( x ) {e}^{- \frac{2 x}{δ}} dx , \qquad ( 2 )$$

$$with$$

$$n ( x ) = \{ \begin{matrix}{n}_{p} , 0 < x \leq {d}_{p} \\ {n}_{t} , {d}_{p} < x \leq {d}_{p} + {d}_{t} \\ {n}_{a} , x > {d}_{p} + {d}_{t}\end{matrix} ,$$

where nₚ is RI of ACE2 layer, nₜ is RI of NL63 viral particle layer, nₐ is RI of air, dₚ is the thickness of ACE2 layer, and dₜ is the thickness of NL63 layer. Based on the ACE2 immobilization,

$$Δ {λ}_{ACE 2} = S ( {n}_{p} - {n}_{a} ) ( 1 - {e}^{- \frac{2 {d}_{p}}{δ}} ) , \qquad ( 3 )$$

where Δλ_{ACE2}=10±1 nm, S=210±10 nm/RIU, nₚ=1.45, nₐ=1, dₚ=5 nm [22], then δ can be estimated to be 89.4 nm, which is similar to the value reported in Ag nanohole array [39]. When the sensor captures the virus, Δλ can be written as,

$$Δ λ = S ( {n}_{t} - {n}_{a} ) {e}^{- \frac{2 {d}_{p}}{δ}} ( 1 - {e}^{- \frac{2 {d}_{t}}{δ}} ) . \qquad ( 4 )$$

Therefore, the effective RI of the virus layer can be estimated as,

$${n}_{t} = \frac{Δ {λ}_{lspr}}{{Se}^{- \frac{2 {d}_{p}}{δ}} ( 1 - {e}^{- \frac{2 {d}_{t}}{δ}} )} + {n}_{a} . \qquad ( 5 )$$

Here we take dₜ=100 nm, since the transmission electron microscopic (TEM) studies of NL63 infected LLCMK2 cells revealed that virions were spherical, spiked, and range from 75 to 115 nm in diameter [40]. Based on FIG. 4D, the effective nₜ versus C_{NL63} is estimated, as plotted in FIG. 18A. The viral layer is a porous layer, not totally covered by the virus particles. Its RI can be estimated by the effective medium theory, the Bruggeman's equation [41],

$${δ}_{v} ( \frac{{ε}_{v} - {ε}_{t}}{{ε}_{v} + {ε}_{t}} ) + ( 1 - {δ}_{v} ) ( \frac{{ε}_{a} - {ε}_{t}}{{ε}_{a} + {ε}_{t}} ) = 0 , \qquad ( 6 )$$

where εₜ, εᵥ and εₐ are the effective dielectric constants of the medium, the dielectric constant of the NL63 virus, and the dielectric constant of air, respectively, and δᵥ is the volume fraction of the NL63. Also, εₜ=nₜ², δᵥ can be written as

$${δ}_{v} = \frac{( \frac{{n}_{a}^{2} - {n}_{t}^{2}}{{n}_{a}^{2} + {n}_{t}^{2}} )}{( \frac{{n}_{a}^{2} - {n}_{t}^{2}}{{n}_{a}^{2} + {n}_{t}^{2}} ) - ( \frac{{n}_{v}^{2} - {n}_{t}^{2}}{{n}_{v}^{2} + {n}_{t}^{2}} )} . \qquad ( 7 )$$

The experimentally obtained δᵥ versus C_{NL63} is plotted in FIG. 18B. If the NL63 viral particle is assumed to be a sphere, then the number of viral particles that are detectable on each individual AgNT can be estimated based on FIG. 4D. As shown in FIG. 5B, at the LOD, every AgNT has an average of one viral particle bonded. At the highest detectable concentration (10⁵ PFU/mL), each AgNT has an average 12 virus particles bonded. According to the area of an AgNT, the total number of virus particles available on an AgNT is estimated to be around 32. So, there is still room for the sensor to detect higher virus concentrations.

### The Time-Dependent Detection of Spike RBD and NL63:

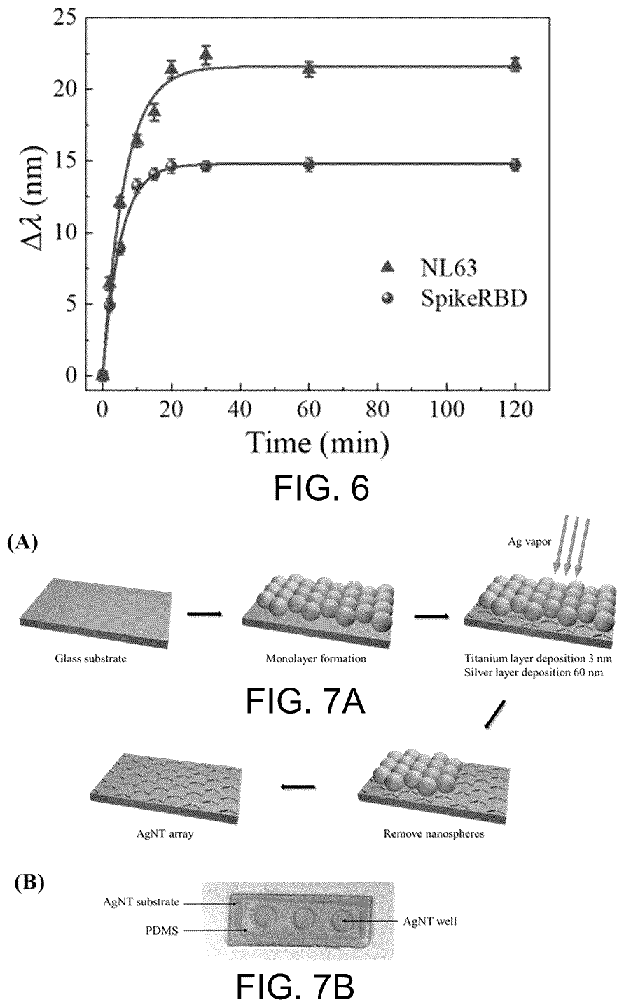

The detection time of AgNT array sensor is mainly determined by the ACE2-SpikeRBD or ACE2-virus binding time. To assess the real detection time for the AgNT LSPR sensor, systematic investigations have been performed. First, the AgNT array sensor was incubated with 20 μL spikeRBD at C_{spikeRBD}=2020 pM for varied time duration t. The purple circles in the FIG. 6 plot Δλ_{spikeRBD} as a function of t. When t<20 min, Δλ_{spikeRBD} red shifts almost monotonically with t, while when t≥20 min, the Δλ_{spikeRBD} reaches a saturation value, Δλ_{spikeRBD}^{S}=14.7±0.5 nm. At t=5 min, a distinct Δλ_{spikeRBD} (=8.9±0.5 nm) can be observed, while at t=10 min, Δλ_{spikeRBD} (=13.3±0.5 nm) is near 90% of the saturation Δλ_{spikeRBD}^{S}. Such a trend is consistent with molecular binding kinetics on a surface, which can be written as

$$Δ λ = Δ {λ}^{S} ( 1 - {e}^{- \frac{t}{{{t}^{0}}^{}}} ) , \qquad ( 8 )$$

where Δλ^{s} is the saturation wavelength shift and t⁰ is the binding time constant. For spike RBD protein detection at C_{spikeRBD}=2020 pM, Δλ_{spikeRBD}^{S} can be obtained as 14.8±0.2 nm, and t_{spikeRBD}⁰ is 5.1±0.2 min. Similarly, the AgNT array sensor was incubated with 20 μL NL63 at C_{NL63}=12500 PFU/mL for various t. The red triangles in the FIG. 6 plot Δλ_{NL63} as a function of t, and a trend similar to that of Δλ_{spikeRBD-t} is observed. Based on Eq. 8, Δλ_{NL63}^{S}=21.6±0.4 nm and t_{NL63}⁰=6.4±0.4 min are obtained. The t_{spikeRBD}⁰ is slightly smaller than t_{NL63}⁰, indicating that spike RBD protein of SARS-COV-2 binds faster to ACE2. Both results indicate that the AgNT based sensor has a detection time of less than 20 min.

### CONCLUSIONS

In summary, an ACE2 functionalized AgNT array LSPR sensor has been developed and shown to have a high specificity to SARS-COV-2 and NL63. The LSPR sensor can detect the SARS-CoV-2 spike RBD protein and NL63 virus with high sensitivity and selectivity. For all these detections, the changes of transmission intensity at λ=750 nm and the shift of the LSPR wavelength λ₀ follow a linear relationship with the log[C_{spikeRBD}] or log[C_{NL63}]. For the spike RBD protein, the detection is observed in the concentration region from 0.38 pM to 9420 pM. The LOD is 0.38 pM. For NL 63, the detection range is from 625 to 10⁴ PFU/mL in untreated saliva, with a LOD of 625 PFU/mL. The detection time is governed by the binding time of ACE2 and spike RBD protein, and systematical experiments have shown it to be <20 min for both spike RBD protein and NL63 detections. This LSPR sensor configuration is very simple and many of the measurements can be performed using a handheld UV-Vis spectrometer (Ocean Optics USB2000). In principle, any LSPR sensor can adopt the proposed strategy in FIG. 1 for spike RBD protein or coronavirus detection.

In fact, most works reported in the literature (see Table S1) are concentrated on spike protein detection, and only few really report the results on real virus detection [42-44]. Among them, only four works focused on plasmonic based sensors [12-14, 45]. Though the LOD of our sensor on spike RBD detection is significantly higher than the graphene-based field-effect transistor sensor and cell-based sensor (but lower than that of the electrochemical immunoassay [47]), the fabrication and detection instrument for our sensors are much more simplified and cost effective. For the virus sensing, the LOD of our sensor is comparable to or better that of the plasmonic sensor and the electrochemical immunoassay [48]. In addition, the viral particles used in Refs. and are SARS-COV-2 pseudotyped viral particles or inactivated virus, not the real virus. Our estimation on the number of viral particle binding shows that at the LOD, every AgNT has an average of one viral particle bonded, which further confirms the reliability of our detection.

Clearly this AgNT sensor has the following advantages: first, the fabrication procedure for AgNT array is straightforward and inexpensive; second, the AgNT array can be fabricated into a large and uniform area; and finally, the sensor measurement can be fulfilled by a handheld UV-Vis spectrometer. In addition, AgNT should have a higher plasmonic effect compared to Au. However, the structure of the AgNT is not optimized for sensitivity. By varying the composition and size of the AgNT, one could significantly improve the sensitivity of the LSPR sensor from ˜210 nm/RIU to ˜700 nm/RIU or possibly better [50, 51], thus lowering the LOD. With improvements, a fast and cost-effective optical sensor can be expected as a potential point-of-care SARS-COV-2 diagnostic platform for sample analysis.

### REFERENCES FOR EXAMPLE 1

[1] B. Udugama, P. Kadhiresan, H. N. Kozlowski, A. Malekjahani, M. Osborne, V. Y. C. Li, et al., Diagnosing COVID-19: The Disease and Tools for Detection, ACS Nano, 14 (2020) 3822-35.
[2] T. Nolan, R. E. Hands, S. A. Bustin, Quantification of mRNA using real-time RT-PCR, Nature Protocols, 1 (2006) 1559-82.
[3] M. Alafeef, K. Dighe, P. Moitra, D. Pan, Rapid, Ultrasensitive, and Quantitative Detection of SARS-COV-2 Using Antisense Oligonucleotides Directed Electrochemical Biosensor Chip, ACS Nano, 14 (2020) 17028-45.
[4] N. Ravi, D. L. Cortade, E. Ng, S. X. Wang, Diagnostics for SARS-COV-2 detection: A comprehensive review of the FDA-EUA COVID-19 testing landscape, Biosensors and Bioelectronics, 165 (2020) 112454.
[5] Z. Li, Y. Yi, X. Luo, N. Xiong, Y. Liu, S. Li, et al., Development and clinical application of a rapid IgM-IgG combined antibody test for SARS-COV-2 infection diagnosis, Journal of Medical Virology, 92 (2020) 1518-24.
[6] A. Alekseenko, D. Barrett, Y. Pareja-Sanchez, R. J. Howard, E. Strandback, H. Ampah-Korsah, et al., Direct detection of SARS-COV-2 using non-commercial RT-LAMP reagents on heat-inactivated samples, Scientific Reports, 11 (2021) 1820.
[7] M. E. Stewart, C. R. Anderton, L. B. Thompson, J. Maria, S. K. Gray, J. A. Rogers, et al., Nanostructured Plasmonic Sensors, Chemical Reviews, 108 (2008) 494-521.
[8] K. M. Mayer, J. H. Hafner, Localized Surface Plasmon Resonance Sensors, Chemical Reviews, 111 (2011) 3828-57.
[9] M. Li, S. K. Cushing, N. Wu, Plasmon-enhanced optical sensors: a review, Analyst, 140 (2015) 386-406.
[10] P. Moitra, M. Alafeef, K. Dighe, M. B. Frieman, D. Pan, Selective Naked-Eye Detection of SARS-COV-2 Mediated by N Gene Targeted Antisense Oligonucleotide Capped Plasmonic Nanoparticles, ACS Nano, 14 (2020) 7617-27.
[11] G. Qiu, Z. Gai, Y. Tao, J. Schmitt, G. A. Kullak-Ublick, J. Wang, Dual-Functional Plasmonic Photothermal Biosensors for Highly Accurate Severe Acute Respiratory Syndrome Coronavirus 2 Detection, ACS Nano, 14 (2020) 5268-77.
[12] B. D. Ventura, M. Cennamo, A. Minopoli, R. Campanile, S. B. Censi, D. Terracciano, et al., Colorimetric Test for Fast Detection of SARS-COV-2 in Nasal and Throat Swabs, ACS Sensors, 5 (2020) 3043-8.
[13] S. Aithal, S. Mishriki, R. Gupta, R. P. Sahu, G. Botos, S. Tanvir, et al., SARS-COV-2 detection with aptamer-functionalized gold nanoparticles, Talanta, 236 (2022) 122841.
[14] L. Huang, L. Ding, J. Zhou, S. Chen, F. Chen, C. Zhao, et al., One-step rapid quantification of SARS-COV-2 virus particles via low-cost nanoplasmonic sensors in generic microplate reader and point-of-care device, Biosensors and Bioelectronics, 171 (2021) 112685.
[15] T. T. S. Lew, K. M. M. Aung, S. Y. Ow, S. N. Amrun, L. Sutarlie, L. F. P. Ng, et al., Epitope-Functionalized Gold Nanoparticles for Rapid and Selective Detection of SARS-COV-2 IgG Antibodies, ACS Nano, 15 (2021) 12286-97.
[16] R. Funari, K.-Y. Chu, A. Q. Shen, Detection of antibodies against SARS-COV-2 spike protein by gold nanospikes in an opto-microfluidic chip, Biosensors and Bioelectronics, 169 (2020) 112578.
[17] A. N. Masterson, B. B. Muhoberac, A. Gopinadhan, D. J. Wilde, F. T. Deiss, C. C. John, et al., Multiplexed and High-Throughput Label-Free Detection of RNA/Spike Protein/IgG/IgM Biomarkers of SARS-COV-2 Infection Utilizing Nanoplasmonic Biosensors, Analytical Chemistry, 93 (2021) 8754-63.
[18] K. Khurana, N. Jaggi, Localized Surface Plasmonic Properties of Au and Ag Nanoparticles for Sensors: a Review, Plasmonics, 16 (2021) 981-99.
[19] W. M. Ingram, C. Han, Q. Zhang, Y. Zhao, Optimization of Ag-Coated Polystyrene Nanosphere Substrates for Quantitative Surface-Enhanced Raman Spectroscopy Analysis, J Phys Chem C, 119 (2015) 27639-48.
[20] A. Tripp Ralph, D. Moore, L. Jones, W. Sullender, J. Winter, J. Anderson Larry, Respiratory Syncytial Virus G and/or SH Protein Alters Th1 Cytokines, Natural Killer Cells, and Neutrophils Responding to Pulmonary Infection in BALB/c Mice, Journal of Virology, 73 (1999) 7099-107.
[21] A. J. Haes, R. P. Van Duyne, A Nanoscale Optical Biosensor: Sensitivity and Selectivity of an Approach Based on the Localized Surface Plasmon Resonance Spectroscopy of Triangular Silver Nanoparticles, Journal of the American Chemical Society, 124 (2002) 10596-604.
[22] H. Chen, A. Das, L. Bi, N. Choi, J.-I. Moon, Y. Wu, et al., Recent advances in surface-enhanced Raman scattering-based microdevices for point-of-care diagnosis of viruses and bacteria, Nanoscale, 12 (2020) 21560-70.
[23] J. Babcock Gregory, J. Esshaki Diana, D. Thomas William, M. Ambrosino Donna, Amino Acids 270 to 510 of the Severe Acute Respiratory Syndrome Coronavirus Spike Protein Are Required for Interaction with Receptor, Journal of Virology, 78 (2004) 4552-60.
[24] J. X. Fu, A. Collins, Y. P. Zhao, Optical Properties and Biosensor Application of Ultrathin Silver Films Prepared by Oblique Angle Deposition, The Journal of Physical Chemistry C, 112 (2008) 16784-91.
[25] J. C. Riboh, A. J. Haes, A. D. McFarland, C. Ranjit Yonzon, R. P. Van Duyne, A Nanoscale Optical Biosensor: Real-Time Immunoassay in Physiological Buffer Enabled by Improved Nanoparticle Adhesion, The Journal of Physical Chemistry B, 107 (2003) 1772-80.
[26] A. Gole, C. Dash, V. Ramakrishnan, S. R. Sainkar, A. B. Mandale, M. Rao, et al., Pepsin-Gold Colloid Conjugates: Preparation, Characterization, and Enzymatic Activity, Langmuir, 17 (2001) 1674-9.
[27] Z. Wang, S. Zong, W. Li, C. Wang, S. Xu, H. Chen, et al., SERS-Fluorescence Joint Spectral Encoding Using Organic-Metal-QD Hybrid Nanoparticles with a Huge Encoding Capacity for High-Throughput Biodetection: Putting Theory into Practice, Journal of the American Chemical Society, 134 (2012) 2993-3000.
[28] G. F. Wang, R. J. Lipert, M. Jain, S. Kaur, S. Chakraboty, M. P. Torres, et al., Detection of the Potential Pancreatic Cancer Marker MUC4 in Serum Using Surface-Enhanced Raman Scattering, Analytical Chemistry, 83 (2011) 2554-61.
[29] S. Zhang, A. Garcia-D′Angeli, J. P. Brennan, Q. Huo, Predicting detection limits of enzyme-linked immunosorbent assay (ELISA) and bioanalytical techniques in general, Analyst, 139 (2014) 439-45.
[30] A. Yakimovich, V. Andriasyan, R. Witte, I. H. Wang, V. Prasad, M. Suomalainen, et al., Plaque2.0—A High-Throughput Analysis Framework to Score Virus-Cell Transmission and Clonal Cell Expansion, PLOS One, 10 (2015) e0138760.
[31] S. J. Martin, The biochemistry of viruses: CUP Archive; 1978.
[32] E. Carpenter John, P. Henderson Ernesto, C. Grose, Enumeration of an Extremely High Particle-to-PFU Ratio for Varicella-Zoster Virus, Journal of Virology, 83 (2009) 6917-21.
[33] H. W. Despres, M. G. Mills, D. J. Shirley, M. M. Schmidt, M.-L. Huang, K. R. Jerome, et al., Quantitative measurement of infectious virus in SARS-COV-2 Alpha, Delta and Epsilon variants reveals higher infectivity (viral titer: RNA ratio) in clinical samples containing the Delta and Epsilon variants, medRxiv, (2021) 2021.09.07.21263229.
[34] E. Özkumur, J. W. Needham, D. A. Bergstein, R. Gonzalez, M. Cabodi, J. M. Gershoni, et al., Label-free and dynamic detection of biomolecular interactions for high-throughput microarray applications, Proceedings of the National Academy of Sciences, 105 (2008) 7988.
[35] J. Piehler, A. Brecht, G. Gauglitz, Affinity Detection of Low Molecular Weight Analytes, Analytical Chemistry, 68 (1996) 139-43.
[36] A. M. Hamed, Image processing of corona virus using interferometry, Optics and Photonics Journal, 6 (2016) 75.
[37] Y. Li, Plasmonic optics: theory and applications, Washington, USA: SPIE; 2017.
[38] L. S. Jung, C. T. Campbell, T. M. Chinowsky, M. N. Mar, S. S. Yee, Quantitative Interpretation of the Response of Surface Plasmon Resonance Sensors to Adsorbed Films, Langmuir, 14 (1998) 5636-48.
[39] B. Ai, P. Basnet, S. Larson, W. Ingram, Y. Zhao, Plasmonic sensor with high figure of merit based on differential polarization spectra of elliptical nanohole array, Nanoscale, 9 (2017) 14710-21.
[40] J. M. Orenstein, B. Banach, S. C. Baker, Morphogenesis of Coronavirus HCoV-NL63 in Cell Culture: A Transmission Electron Microscopic Study, Open Infect Dis J, 2 (2008) 52-8.
[41] R. Landauer, Electrical conductivity in inhomogeneous media, AIP Conference Proceedings, 40 (1978) 2-45.
[42] G. Seo, G. Lee, M. J. Kim, S.-H. Baek, M. Choi, K. B. Ku, et al., Rapid Detection of COVID-19 Causative Virus (SARS-COV-2) in Human Nasopharyngeal Swab Specimens Using Field-Effect Transistor-Based Biosensor, ACS Nano, 14 (2020) 5135-42.
[43] S. Kim, H. Ryu, S. Tai, M. Pedowitz, J. R. Rzasa, D. J. Pennachio, et al., Real-time ultra-sensitive detection of SARS-COV-2 by quasi-freestanding epitaxial graphene-based biosensor, Biosensors and Bioelectronics, 197 (2022) 113803.
[44] D. K. Agarwal, V. Nandwana, S. E. Henrich, V. P. V. N. Josyula, C. S. Thaxton, C. Qi, et al., Highly sensitive and ultra-rapid antigen-based detection of SARS-COV-2 using nanomechanical sensor platform, Biosensors and Bioelectronics, 195 (2022) 113647.
[45] N. Cennamo, L. Pasquardini, F. Arcadio, L. Lunelli, L. Vanzetti, V. Carafa, et al., SARS-COV-2 spike protein detection through a plasmonic D-shaped plastic optical fiber aptasensor, Talanta, 233 (2021) 122532.
[46] S. Mavrikou, G. Moschopoulou, V. Tsekouras, S. Kintzios, Development of a Portable, Ultra-Rapid and Ultra-Sensitive Cell-Based Biosensor for the Direct Detection of the SARS-COV-2 S1 Spike Protein Antigen, Sensors, 20 (2020) 3121.
[47] L. Fabiani, M. Saroglia, G. Galatà, R. De Santis, S. Fillo, V. Luca, et al., Magnetic beads combined with carbon black-based screen-printed electrodes for COVID-19: A reliable and miniaturized electrochemical immunosensor for SARS-COV-2 detection in saliva, Biosensors and Bioelectronics, 171 (2021) 112686.
[48] H. Yousefi, A. Mahmud, D. Chang, J. Das, S. Gomis, J. B. Chen, et al., Detection of SARS-COV-2 Viral Particles Using Direct, Reagent-Free Electrochemical Sensing, Journal of the American Chemical Society, 143 (2021) 1722-7.
[49] J. Li, A. Wuethrich, S. Edwardraja, R. J. Lobb, S. Puttick, S. Rose, et al., Amplification-Free SARS-COV-2 Detection Using Nanoyeast-scFv and Ultrasensitive Plasmonic Nanobox-Integrated Nanomixing Microassay, Analytical Chemistry, 93 (2021) 10251-60.
[50] S. Larson, Y. Zhao, Localized Surface Plasmonic Resonance and Sensing Properties of Ag—MgF2 Composite Nanotriangles, The Journal of Physical Chemistry C, 122 (2018) 7374-81.
[51] S. Larson, Z. Yang, Y. Zhao, Improving LSPR sensing performance using multilayered composition graded Ag—Cu nanotriangle arrays, Chemical Communications, 55 (2019) 1342-4.

### Supplemental Information for Example 1

TABLE S2
Receptors of different kinds of coronavirus.

| Coronavirus | Receptor | Ref |
|---|---|---|
| Human coronavirus OC43 (CoV OC43) | N-acetyl-9-O-acetylneuraminic acid | [23] |
| Human coronavirus HKU1 (CoV HKU1) | 9-O-acetylneuraminic acid | [24] |
| Human coronavirus 229E (CoV 229E) | Aminopeptidase N (hAPN [CD13]) | [25] |
| Human coronavirus NL63 (CoV NL63) | ACE2 | [23] |
| Middle East respiratory syndrome-related | Dipeptidyl peptidase-4 | [23] |
| coronavirus (MERS-CoV) | | |
| Severe acute respiratory syndrome | ACE2 | [26] |
| coronavirus (SARS-CoV) | | |
| Severe respiratory syndrome | ACE2 | [27] |
| coronavirus 2 (SARS-CoV-2) | | |

### S1. Optimize the Buffer for ACE2 Protein Immobilization on the AgNT Array:

To achieve specific detection of spike RBD protein and NL63, ACE2 protein is used to functionalize the AgNT array. As previously shown, Ag is not chemically stable in NaCl solution [28]. Since ACE2 protein is supplied in PBS, the effect of PBS and other buffers on the LSPR response of AgNT has been investigated before ACE2 immobilization. When incubating AgNT array with PBS buffer, the λ₀ blue-shifted significantly, from 641 nm to 542 nm (FIG. 10), indicating that the actual size of the AgNTs became smaller. Thus, PBS buffer is not suitable for immobilizing ACE2 protein on the AgNT array. Phosphate buffer is a chloride-free buffer, and when incubating with AgNT array, the λ₀ almost did not change (FIG. 10), indicating that this buffer can be used for ACE2 immobilization. Therefore, phosphate buffer has been used to dialyze the ACE2 solution to remove the chloride ions.

### S2. Surface Enhanced Raman Scattering Characterization of ACE2 Immobilization on the AgNR Substrate:

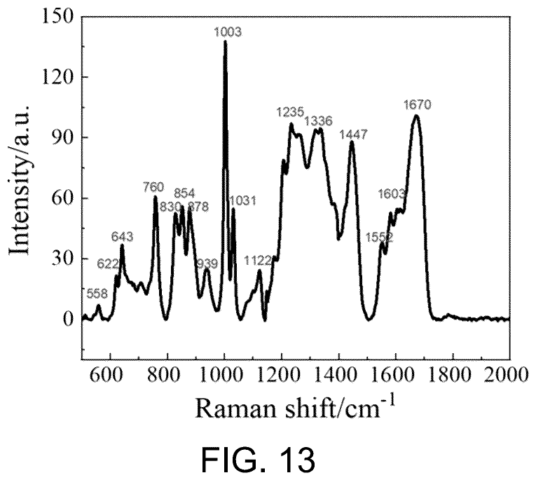

An alternative way to characterize the ACE2 protein adsorption on the silver surface is using surface enhanced Raman scattering (SERS) spectroscopy. AgNR arrays prepared by the oblique angle deposition (OAD) are excellent SERS substrates as described previously [29]. Briefly, clean glass slides (0.5 inch×0.5 inch) were loaded into a vacuum deposition chamber with the substrate normal antiparallel to the incident vapor direction. Firstly, 20 nm Ti and 200 nm Ag films were deposited in sequence at a rate of 0.2 nm/s and 0.3 nm/s, respectively. Then, the substrate normal was rotated to 86° relative to the incident vapor direction, and a thickness of 2000 nm Ag film was then deposited at a rate of 0.3 nm/s to fabricate the arrayed AgNRs. The entire evaporation process was conducted under a high vacuum condition (<3×10⁻⁶ Torr). Subsequently, a PDMS layer with arrayed small wells (2×2 wells, well diameter of 4 mm, depth of 1 mm) was molded on the AgNRs substrate (AgNR well). ACE2 protein was immobilized on the AgNR arrays SERS substrate using the same manner as the immobilization on the AgNT: 20 μL ACE2 solution (66 μg/mL) was added into each AgNR well and incubated for 2 h at room temperature. Then, the wells were washed with DI water and air-dried for SERS measurements. The SERS measurements were performed using a confocal Raman microscope (Renishaw InVia) with a 785 nm excitation laser and 20× objective lens. The laser power was 9 mW and the acquisition time was 1 s. An averaged SERS spectrum was obtained via 30 SERS scans which were taken at different positions (N=30), and is shown in FIG. 13. The spectrum was baseline subtracted using the software Wire 4.3. The SERS characterization peaks are identified in FIG. 13, and the assignments are summarized in Table S3. Most peaks can be assigned to amino acids of protein, C—N and C—H vibration mode [30], which demonstrates the successfully assembly of ACE2 protein on the silver surface.

TABLE S3
SERS peak assignment for ACE2 protein.

| Peak (cm⁻¹) | Assignment |
|---|---|
| 558 | S—S |
| 643 | Tyr |
| 760 | Trp |
| 830 | Tyr |
| 854 | Tyr |
| 878 | Trp |
| 939 | N—C_{α}—C |
| 1003 | Phe |
| 1031 | Phe |
| 1122 | C—N |
| 1235 | Amide III |
| 1336 | Trp, Cₐ—H (def) |
| 1447 | C—H (def) |
| 1552 | Indole ring (Trp) |
| 1603 | Tyr, Trp, Phe |
| 1670 | Amide I |

### S3. Reproducibility of AgNT Array Sensor:

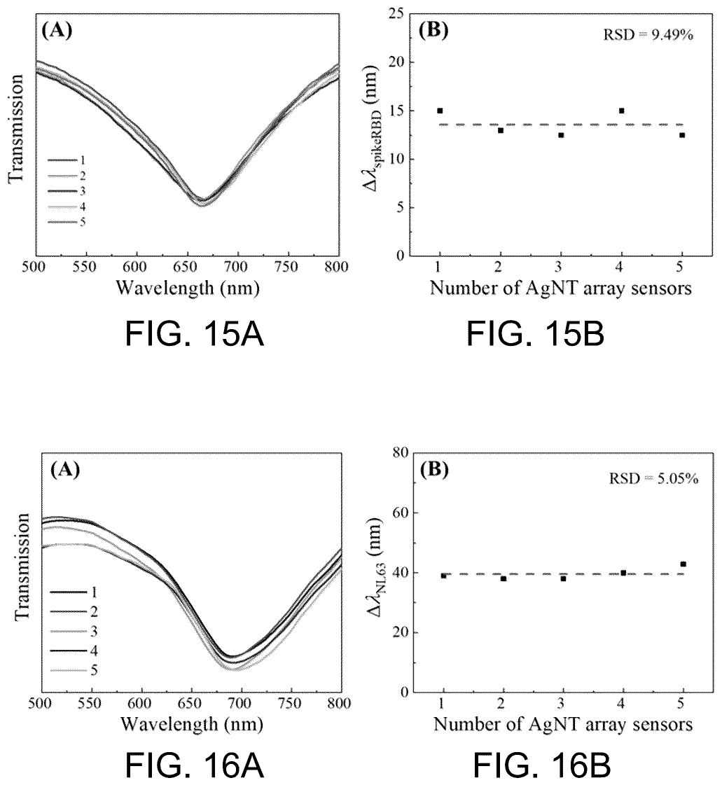

To characterize reproducibility of the AgNT array sensors, five batches of the ACE2-AgNT sensors were prepared in the same manner and then utilized to detect spike RBD protein and CoV NL63. The transmission spectra of the 5 ACE2-AgNT array sensors treated by the spike RBD protein of C_{spikeRBD}=2020 pM are shown in FIG. 15A, and their corresponding LSPR shifts Δλ_{spikeRBD} are plotted in FIG. 15B. The spectra dips are almost overlap with each other while a small relative standard deviation (RSD)<10% is shown in Δλ_{spikeRBD}. Similarly, the transmission spectra of the 5 sensors treated by CoV NL63 of 50000 PFU/mL are shown in FIG. S16A, and their corresponding LSPR shifts Δλ_{NL63} are plotted in FIG. 16B. A small RSD of 5.05% is also observed in Δλ_{NL63}. These characterizations indicate that the AgNT array sensors possess good reproducibility.

TABLE S4
The number of spike RBD protein molecules in the
light beam during the detection via an AgNT well.

| Spike RBD | Spike RBD | Spike RBD protein |
|---|---|---|
| Concentration | protein molecules | molecules in the |
| (pM) | in the wells | light path (1 mm²) |
| 0.377 | 4.54 × 10⁶ | 3.61 × 10⁵ |
| 2.03 | 2.45 × 10⁷ | 1.95 × 10⁶ |
| 6.78 | 8.17 × 10⁷ | 6.50 × 10⁶ |
| 13.6 | 1.63 × 10⁸ | 1.30 × 10⁷ |
| 67.4 | 8.12 × 10⁸ | 6.47 × 10⁷ |
| 135 | 1.62 × 10⁹ | 1.29 × 10⁸ |
| 674 | 8.12 × 10⁹ | 6.47 × 10⁸ |
| 2.02 × 10³ | 2.43 × 10¹⁰ | 1.94 × 10⁹ |
| 9.42 × 10³ | 1.13 × 10¹¹ | 9.03 × 10⁹ |

### Section S4. Modeling for Virus Detection:

The effective refractive index is estimated via an integration of the local refractive index from the surface of AgNT (x=0) to infinity and is presented as Eq. 3 in the main text. The effective refractive index can be written as, [31, 32]

$${n}_{eff} = \frac{2}{δ} [ {\int }_{0}^{{d}_{p}} {n}_{p} + {\int }_{{d}_{p}}^{{d}_{p} + {d}_{t}} {n}_{t} + {\int }_{{d}_{p} + {d}_{t}}^{∞} {n}_{a} ] {e}^{- \frac{2 x}{δ}} dx = {n}_{p} - {n}_{p} {e}^{- \frac{2 {d}_{p}}{δ}} + {n}_{t} {e}^{- \frac{2 {d}_{p}}{δ}} - {n}_{t} {e}^{- \frac{2 {d}_{p} + 2 {d}_{t}}{δ}} + {n}_{a} {e}^{- \frac{2 {d}_{p} + 2 {d}_{t}}{δ} .} \qquad ( S1 )$$

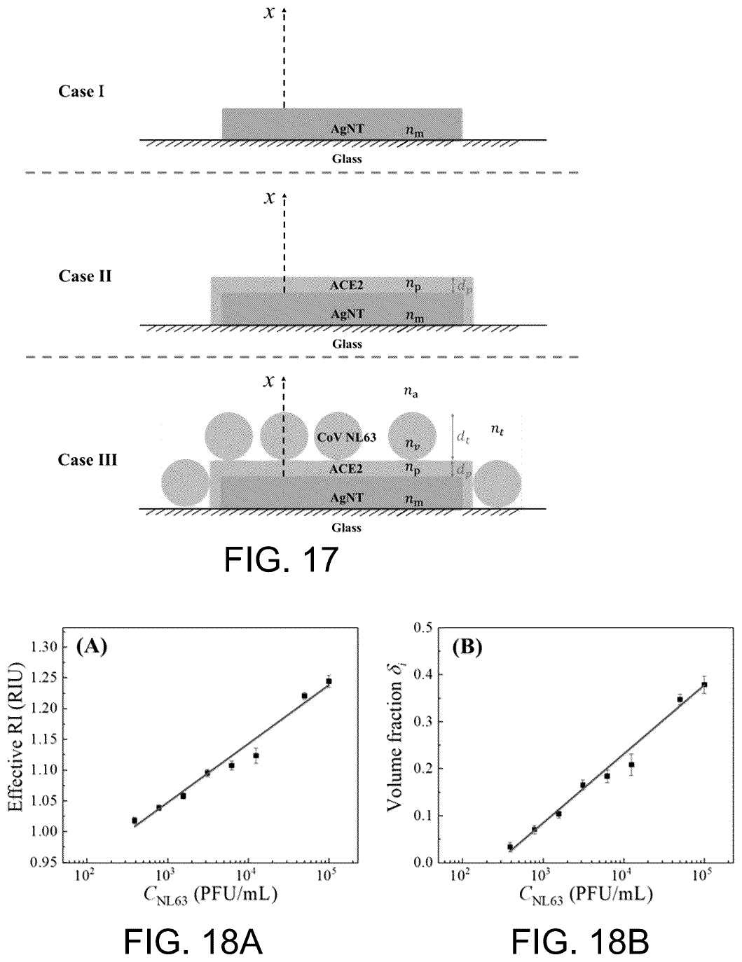

As shown in FIG. 17, in order to find the Ad, one needs to estimate n_{eff} for three different cases. For Case I, if the outside of the AgNT is only air, the effective refractive index n_{eff}^{I} can be written as,

$${n}_{eff}^{I} = \frac{2}{δ} {\int }_{0}^{∞} {n}_{a} {e}^{- \frac{2 x}{δ}} dx = - {n}_{a} {e}^{- \frac{2 x}{δ}} {\vert}_{0}^{∞} = {n}_{a} . \qquad ( S 2 )$$

For Case II, the outside of the AgNT is coating with ACE2 protein layer, the effective refractive index n_{eff}^{II} can be written as,

$${n}_{eff}^{II} = {n}_{p} - {n}_{p} {e}^{- \frac{2 {d}_{p}}{δ}} + {n}_{a} {e}^{- \frac{2 {d}_{p}}{δ}} , \qquad ( S3 )$$

The change of n_{eff} between Case II and Case I is,

$$Δ {n}_{eff}^{II - I} = {n}_{eff}^{II} - {n}_{eff}^{I} = {n}_{p} - {n}_{p} {e}^{- \frac{2 {d}_{p}}{δ}} + {n}_{a} {e}^{- \frac{2 {d}_{p}}{δ}} - {n}_{a} = {n}_{p} ( 1 - {e}^{- \frac{2 {d}_{p}}{δ}} ) + {n}_{a} ( {e}^{- \frac{2 {d}_{p}}{δ}} - 1 ) = ( {n}_{p} - {n}_{a} ) ( 1 - {e}^{- \frac{2 {d}_{p}}{δ}} ) . \qquad ( S4 )$$

For Case II, after CoV NL63 detection, the effective refractive index nerf can be written as,

$${n}_{eff}^{III} = {n}_{p} - {n}_{p} {e}^{- \frac{2 {d}_{p}}{δ}} + {n}_{t} {e}^{- \frac{2 {d}_{p}}{δ}} - {n}_{t} {e}^{- \frac{2 {d}_{p} + 2 {d}_{t}}{δ}} + {n}_{a} {e}^{{e}^{- \frac{2 {d}_{p} + 2 {d}_{t}}{δ}}} . \qquad ( S5 )$$

The change of n_{eff} between Case III and Case II is,

$$( S6 )$$

$$Δ {n}_{eff}^{III - II} = {n}_{p} - {n}_{p} {e}^{- \frac{2 {d}_{p}}{δ}} + {n}_{t} {e}^{- \frac{2 {d}_{p}}{δ}} - {n}_{t} {e}^{- \frac{2 {d}_{p} + 2 {d}_{t}}{δ}} + {n}_{a} {e}^{- \frac{2 {d}_{p} + 2 {d}_{t}}{δ}} - ( {n}_{p} - {n}_{p} {e}^{- \frac{2 {d}_{p}}{δ}} + {n}_{a} {e}^{- \frac{2 {d}_{p}}{δ}} ) = {n}_{t} {e}^{- \frac{2 {d}_{p}}{δ}} - {n}_{t} {e}^{- \frac{2 {d}_{p} + 2 {d}_{t}}{δ}} + {n}_{a} {e}^{- \frac{2 {d}_{p} + 2 {d}_{t}}{δ}} - {n}_{a} {e}^{- \frac{2 {d}_{p}}{δ}} = ( {n}_{t} - {n}_{a} ) {e}^{- \frac{2 {d}_{p}}{δ}} ( 1 - {e}^{- \frac{2 {d}_{t}}{δ}} ) .$$

Based on FIG. 4D in main test and Eqs. S5 and S6, the effective nₜ at different C_{NL63} can be extracted and plotted in FIG. 18A as a function of C_{NL63}. It clearly follows a linear relationship in the semi-log plot. Based on the Bruggeman's equation, the experimentally obtained δ_{V} versus C_{NL63} is plotted in FIG. 18B, and it also follows a semi-log relationship.

### Section S5. Estimation of the Full Coverage of Virus on a Single AgNT:

According to the AFM image, the AgNT is an equilateral nanotriangle with the length lₜ=152±3 nm and the height hₜ=60±1 nm. The thickness of the ACE2 protein layer is dₚ=5 nm, and that of CoV NL63 layer is dₜ=100 nm. Therefore, after ACE2 immobilization, the length the NT lₚ=157 nm and the height hₚ=65 nm. If the surface is then covered with a full layer of CoV NL63 layer, then the length of AgNT lₙ=355 nm, and the height hₙ=165 nm. The volume of the CoV NL63 layer can be calculated by

$${V}_{t} = \frac{\sqrt{3}}{2} {h}_{n} {l}_{n}^{2} - \frac{\sqrt{3}}{2} {h}_{p} {l}_{p}^{2} = 1.68 \times 1 {0}^{7} {nm}^{3} .$$

Assuming that a viral CoV NL63 particle has a spherical shape, the volume V_{NL63} of a single CoV NL63 virus is estimated as

$${V}_{NL 63} = \frac{4}{3} {π ( \frac{{d}_{t}}{2} )}^{3} = 5.23 \times {10}^{5} {nm}^{3} .$$

Therefore, the maximum number of CoV NL63 on a single AgNT is estimated to be 32.

### References for Supplemental Information for Example 1

[1] J.-H. Lee, M. Choi, Y. Jung, S. K. Lee, C.-S. Lee, J. Kim, et al., A novel rapid detection for SARS-CoV-2 spike 1 antigens using human angiotensin converting enzyme 2 (ACE2), Biosensors and Bioelectronics, 171 (2021) 112715.
[2] G. Seo, G. Lee, M. J. Kim, S.-H. Baek, M. Choi, K. B. Ku, et al., Rapid Detection of COVID-19 Causative Virus (SARS-COV-2) in Human Nasopharyngeal Swab Specimens Using Field-Effect Transistor-Based Biosensor, ACS Nano, 14 (2020) 5135-42.
[3] P. Fathi-Hafshejani, N. Azam, L. Wang, M. A. Kuroda, M. C. Hamilton, S. Hasim, et al., Two-Dimensional-Material-Based Field-Effect Transistor Biosensor for Detecting COVID-19 Virus (SARS-COV-2), ACS Nano, 15 (2021) 11461-9.
[4] D. K. Agarwal, V. Nandwana, S. E. Henrich, V. P. V. N. Josyula, C. S. Thaxton, C. Qi, et al., Highly sensitive and ultra-rapid antigen-based detection of SARS-COV-2 using nanomechanical sensor platform, Biosensors and Bioelectronics, 195 (2022) 113647.
[5] J. Deng, F. Tian, C. Liu, Y. Liu, S. Zhao, T. Fu, et al., Rapid One-Step Detection of Viral Particles Using an Aptamer-Based Thermophoretic Assay, Journal of the American Chemical Society, 143 (2021) 7261-6.
[6] S. Mavrikou, G. Moschopoulou, V. Tsekouras, S. Kintzios, Development of a Portable, Ultra-Rapid and Ultra-Sensitive Cell-Based Biosensor for the Direct Detection of the SARS-COV-2 S1 Spike Protein Antigen, Sensors, 20 (2020) 3121.
[7] S. Kim, H. Ryu, S. Tai, M. Pedowitz, J. R. Rzasa, D. J. Pennachio, et al., Real-time ultra-sensitive detection of SARS-COV-2 by quasi-freestanding epitaxial graphene-based biosensor, Biosensors and Bioelectronics, 197 (2022) 113803.
[8] L. Shi, L. Wang, X. Ma, X. Fang, L. Xiang, Y. Yi, et al., Aptamer-Functionalized Nanochannels for One-Step Detection of SARS-COV-2 in Samples from COVID-19 Patients, Analytical Chemistry, 93 (2021) 16646-54.
[9] A. Roberts, S. Mahari, D. Shahdeo, S. Gandhi, Label-free detection of SARS-COV-2 Spike S1 antigen triggered by electroactive gold nanoparticles on antibody coated fluorine-doped tin oxide (FTO) electrode, Analytica Chimica Acta, 1188 (2021) 339207.
[10] L. Fabiani, M. Saroglia, G. Galatà, R. De Santis, S. Fillo, V. Luca, et al., Magnetic beads combined with carbon black-based screen-printed electrodes for COVID-19: A reliable and miniaturized electrochemical immunosensor for SARS-COV-2 detection in saliva, Biosensors and Bioelectronics, 171 (2021) 112686.
[11] H. Yousefi, A. Mahmud, D. Chang, J. Das, S. Gomis, J. B. Chen, et al., Detection of SARS-COV-2 Viral Particles Using Direct, Reagent-Free Electrochemical Sensing, Journal of the American Chemical Society, 143 (2021) 1722-7.
[12] R. M. Torrente-Rodríguez, H. Lukas, J. Tu, J. Min, Y. Yang, C. Xu, et al., SARS-COV-2 RapidPlex: A Graphene-Based Multiplexed Telemedicine Platform for Rapid and Low-Cost COVID-19 Diagnosis and Monitoring, Matter, 3 (2020) 1981-98.
[13] S. A. Hashemi, N. G. Golab Behbahan, S. Bahrani, S. M. Mousavi, A. Gholami, S. Ramakrishna, et al., Ultra-sensitive viral glycoprotein detection NanoSystem toward accurate tracing SARS-COV-2 in biological/non-biological media, Biosensors and Bioelectronics, 171 (2021) 112731.
[14] P. K. Sharma, E.-S. Kim, S. Mishra, E. Ganbold, R.-S. Seong, A. K. Kaushik, et al., Ultrasensitive and Reusable Graphene Oxide-Modified Double-Interdigitated Capacitive (DIDC) Sensing Chip for Detecting SARS-COV-2, ACS Sensors, 6 (2021) 3468-76.
[15] S. Peinetti Ana, J. Lake Ryan, W. Cong, L. Cooper, Y. Wu, Y. Ma, et al., Direct detection of human adenovirus or SARS-COV-2 with ability to inform infectivity using DNA aptamer-nanopore sensors, Science Advances, 7 (2021) eabh2848.
[16] R. L. Pinals, F. Ledesma, D. Yang, N. Navarro, S. Jeong, J. E. Pak, et al., Rapid SARS-COV-2 Spike Protein Detection by Carbon Nanotube-Based Near-Infrared Nanosensors, Nano Letters, 21 (2021) 2272-80.
[17] J. Li, A. Wuethrich, S. Edwardraja, R. J. Lobb, S. Puttick, S. Rose, et al., Amplification-Free SARS-COV-2 Detection Using Nanoyeast-scFv and Ultrasensitive Plasmonic Nanobox-Integrated Nanomixing Microassay, Analytical Chemistry, 93 (2021) 10251-60.
[18] G. Huang, H. Zhao, P. Li, J. Liu, S. Chen, M. Ge, et al., Construction of Optimal SERS Hotspots Based on Capturing the Spike Receptor-Binding Domain (RBD) of SARS-COV-2 for Highly Sensitive and Specific Detection by a Fish Model, Analytical Chemistry, 93 (2021) 16086-95.
[19] B. D. Ventura, M. Cennamo, A. Minopoli, R. Campanile, S. B. Censi, D. Terracciano, et al., Colorimetric Test for Fast Detection of SARS-COV-2 in Nasal and Throat Swabs, ACS Sensors, 5 (2020) 3043-8.
[20] S. Aithal, S. Mishriki, R. Gupta, R. P. Sahu, G. Botos, S. Tanvir, et al., SARS-COV-2 detection with aptamer-functionalized gold nanoparticles, Talanta, 236 (2022) 122841.
[21] L. Huang, L. Ding, J. Zhou, S. Chen, F. Chen, C. Zhao, et al., One-step rapid quantification of SARS-COV-2 virus particles via low-cost nanoplasmonic sensors in generic microplate reader and point-of-care device, Biosensors and Bioelectronics, 171 (2021) 112685.
[22] N. Cennamo, L. Pasquardini, F. Arcadio, L. Lunelli, L. Vanzetti, V. Carafa, et al., SARS-COV-2 spike protein detection through a plasmonic D-shaped plastic optical fiber aptasensor, Talanta, 233 (2021) 122532.
[23] F. Li, Structure, Function, and Evolution of Coronavirus Spike Proteins, Annual Review of Virology, 3 (2016) 237-61.
[24] R. J. G. Hulswit, Y. Lang, M. J. G. Bakkers, W. Li, Z. Li, A. Schouten, et al., Human coronaviruses OC43 and HKU1 bind to 9-O-acetylated sialic acids via a conserved receptor-binding site in spike protein domain A, Proceedings of the National Academy of Sciences, 116 (2019) 2681.
[25] A. R. Fehr, S. Perlman, Coronaviruses: an overview of their replication and pathogenesis, Coronaviruses, (2015) 1-23.
[26] X.-Y. Ge, J.-L. Li, X.-L. Yang, A. A. Chmura, G. Zhu, J. H. Epstein, et al., Isolation and characterization of a bat SARS-like coronavirus that uses the ACE2 receptor, Nature, 503 (2013) 535-8.
[27] R. Yan, Y. Zhang, Y. Li, L. Xia, Y. Guo, Q. Zhou, Structural basis for the recognition of SARS-CoV-2 by full-length human ACE2, Science, 367 (2020) 1444.
[28] J. X. Fu, A. Collins, Y. P. Zhao, Optical Properties and Biosensor Application of Ultrathin Silver Films Prepared by Oblique Angle Deposition, The Journal of Physical Chemistry C, 112 (2008) 16784-91.
[29] Y. J. Liu, H. Y. Chu, Y. P. Zhao, Silver Nanorod Array Substrates Fabricated by Oblique Angle Deposition: Morphological, Optical, and SERS Characterizations, Journal of Physical Chemistry C, 114 (2010) 8176-83.
[30] A. Rygula, K. Majzner, K. M. Marzec, A. Kaczor, M. Pilarczyk, M. Baranska, Raman spectroscopy of proteins: a review, Journal of Raman Spectroscopy, 44 (2013) 1061-76.
[31] L. S. Jung, C. T. Campbell, T. M. Chinowsky, M. N. Mar, S. S. Yee, Quantitative Interpretation of the Response of Surface Plasmon Resonance Sensors to Adsorbed Films, Langmuir, 14 (1998) 5636-48.
[32] A. J. Haes, R. P. Van Duyne, A Nanoscale Optical Biosensor: Sensitivity and Selectivity of an Approach Based on the Localized Surface Plasmon Resonance Spectroscopy of Triangular Silver Nanoparticles, Journal of the American Chemical Society, 124 (2002) 10596-604.
[33] R. Landauer, Electrical conductivity in inhomogeneous media, AIP Conference Proceedings, 40 (1978) 2-45.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

Many variations and modifications may be made to the above-described embodiments. All such modifications and variations are intended to be included herein within the scope of this disclosure and protected by the following claims.

We claim:

1. A system for detecting a biological agent, comprising:
a white light source;
a UV-Vis-NIR spectrometer; and
a detecting module, wherein the white light source is configured to direct a white light at the detecting module, wherein the UV-Vis-NIR spectrometer is configured to detect a signal corresponding to nanotriangle present in the detecting module;
wherein the detecting module includes a substrate including a plurality of the nanotriangles having a capture agent disposed on the nanotriangle, wherein the capture agent has an affinity for a biological agent, wherein the biological agent is a type of virus or a type of bacteria,
wherein prior to the introduction of sample of fluid to the detecting module, the UV-Vis-NIR spectrometer is configured to detect a first localized surface plasmon resonance (LSPR) signal that corresponds to the nanotriangle having the capture agent, wherein after introduction of the sample of fluid that contains the biological agent, the UV-Vis-NIR spectrometer is configured to detect a second LSPR signal that corresponds to the nanotriangle having the capture agent and the biological agent bonded to the capture agent, wherein the first LSPR signal and the second LSPR signal are different, wherein the difference corresponds to the amount of biological agent present in the sample.

2. The system of claim 1, wherein when the first LSPR signal and the second LSPR signal are the same, the biological agent is not present in the sample.

3. The system of claim 1, wherein the system is configured to detect the presence of the biological agent is about 20 minutes or less from the time the sample is introduced to the detecting module.

4. The system of claim 1, wherein the detecting module is configured to mix the sample.

5. The system of claim 4, wherein the detecting module is configured to mix the sample using a magnetic nanoparticle.

6. The system of claim 1, wherein the system is configured to detect the presence of the biological agent is about 10 minutes or less from the time the sample is introduced to the detecting module.

7. The system of claim 1, wherein the nanotriangle is a silver nanotriangle, a silver composite nanotriangle, a gold nanotriangle, or a gold composite nanotriangle.

8. A device for detecting a biological agent, comprising:
a white light source;
a UV-Vis-NIR spectrometer; and
a detecting module comprising a substrate including a plurality of nanotriangles having a capture agent disposed on the nanotriangle, wherein the capture agent has an affinity for a biological agent, wherein the biological agent is a type of virus or a type of bacteria,
wherein the white light source, the UV-Vis-NIR spectrometer, and the detecting module are interfaced so that the light from the white light source is configured to be directed towards the substrate of the detecting module, wherein the UV-Vis-NIR spectrometer is configured relative to the detecting module to receive a signal from the nanotriangles as a result of the light from the white light source interacting with the nanotriangles.

9. The device of claim 8, wherein the type of virus is a coronavirus.

10. The device of claim 9, wherein the coronavirus is SARS-COV-2 or variant thereof.

11. The device of claim 8, wherein the nanotriangle is a silver nanotriangle, a silver composite nanotriangle, a gold nanotriangle, or a gold composite nanotriangle.

12. The device of claim 11, wherein the nanotriangle has a longest dimension of about 50 nm to 2000 nm and a height of about 10 nm to 100 nm.

13. The device of claim 11, wherein the silver composite nanotriangle or gold composite nanotriangle is a silver-silicon dioxide composite nanotriangle, a silver-magnesium fluoride composite nanotriangle, a silver-titanium dioxide composite nanotriangle, silver-copper composite nanotriangle, silver-gold composite nanotriangle, silver (gold)-aluminum composite nanotriangle, silver-copper composite nanotriangle, or gold-aluminum composite nanotriangle.

14. The device of claim 8, wherein the capture agent is an angiotensin-converting enzyme 2 (ACE-2) protein.

15. The device of claim 8, wherein the capture agent is an enzyme, aptamer, or hapten.

16. The device of claim 8, wherein the device is configured to detect the presence of a biological agent within 20 minutes upon introduction of a sample to the detecting module, wherein the sample includes the biological agent.

17. The device of claim 8, wherein the substrate is an array of the plurality of nanotriangles at addressable locations.

18. The device of claim 8, wherein the UV-Vis-NIR spectrometer is configured to detect a change in the localized surface plasmon resonance (LSPR) of the nanotriangle with the capture agent bound to the nanotriangle and when the biological agent is bonded to the capture agent that is bonded to the nanotriangle.

19. A method of detecting a biological agent, wherein the method includes:
disposing a sample of a fluid onto an area of the detecting module of claim 1, wherein prior to disposing the sample, the detecting module includes a capture agent bonded to the nanotriangle and wherein a first localized surface plasmon resonance (LSPR) signal is known that corresponds to the nanotriangle bonded to the capture agent; and
detecting a second LSPR signal using the UV-Vis-NIR spectrometer after disposing the sample in the first area, wherein when the second LSPR signal is different than the first LSPR signal, this indicates that the biological agent is bonded to the capture agent, which indicates that the biological agent is present in the sample.

20. The method of claim 19, wherein first LSPR signal and the second LSPR signal are the same if the biological agent is not present, wherein the magnitude of the difference between the first LSPR signal and the second LSPR signal corresponds to the amount of biological agent present in the sample, wherein the method of detecting takes 20 minutes or less.
